(19) United States
(12) Patent Application Publication
(10) Pub. No.: US 2009/0062644 A1
(43) Pub. Date: Mar. 5, 2009

(54) SYSTEM AND METHOD FOR ULTRASOUND HARMONIC IMAGING

(76) Inventors: Gerald McMorrow, Redmond, WA (US); FUXING YANG, Woodinville, WA (US); Yanwei Wang, Woodinville, WA (US)

Correspondence Address: BLACK LOWE & GRAHAM, PLLC, 701 FIFTH AVENUE, SUITE 4800, SEATTLE, WA, 98104, US

(21) Appl. No.: 11/968,027

(22) Filed: Dec. 31, 2007

Related U.S. Application Data
(63) Continuation-in-part of application No. 11/213,284, filed on Aug. 26, 2005.
Application No. 11/213,284 is a continuation-in-part of application No. 11/010,539, filed on Dec. 13, 2004.
Application No. 11/010,539 is a continuation-in-part of application No. 10/523,681, filed on Sep. 23, 2005.
Application No. 10/523,681 is a continuation-in-part of application No. 11/625,802, filed on Jan. 22, 2007.
Application No. 11/625,802 is a continuation-in-part of application No. 11/925,843, filed on Oct. 27, 2007.
Application No. 11/925,843 is a continuation-in-part of application No. 11/926,522, filed on Oct. 29, 2007.
Application No. 11/926,522 is a continuation-in-part of application No. 10/704,996, filed on Nov. 10, 2003.
Application No. 10/704,996 is a continuation-in-part of application No. 11/295,043, filed on Dec. 6, 2005.
Application No. 11/295,043 is a continuation-in-part of application No. 11/925,850, filed on Oct. 27, 2007.
Application No. 11/925,850 is a continuation-in-part of application No. 11/119,355, filed on Apr. 29, 2005.
Application No. 11/119,355 is a continuation-in-part of application No. 10/701,955, filed on Nov. 5, 2003, now Pat. No. 7,087,022.
Application No. 10/701,955 is a continuation-in-part of application No. 10/443,126, filed on May 20, 2003, now Pat. No. 7,041,059.
Application No. 10/443,126 is a continuation-in-part of application No. 11/061,867, filed on Feb. 17, 2005.
Application No. 11/061,867 is a continuation-in-part of application No. 10/704,966, filed on Nov. 12, 2003, now Pat. No. 6,803,308.
Application No. 10/704,966 is a continuation-in-part of application No. 10/607,919, filed on Jun. 27, 2003, now Pat. No. 6,884,217.
Application No. 10/607,919 is a continuation-in-part of application No. PCT/US03/24368, filed on Aug. 1, 2003.
Application No. PCT/US03/24368 is a continuation-in-part of application No. PCT/US03/14785, filed on May 9, 2003.
Application No. PCT/US03/14785 is a continuation of application No. 10/165,556, filed on Jun. 7, 2002, now Pat. No. 6,676,605.
Application No. 10/165,556 is a continuation-in-part of application No. 10/888,735, filed on Jul. 9, 2004, now abandoned.
Application No. 10/888,735 is a continuation-in-part of application No. 10/633,186, filed on Jul. 31, 2003, now Pat. No. 7,004,904.
Application No. 10/633,186 is a continuation-in-part of application No. 10/443,126, filed on May 20, 2003, now Pat. No. 7,041,059.
(60) Provisional application No. 60/882,888, filed on Dec. 29, 2006.
(60) Provisional application No. 60/938,446, filed on May 16, 2007.
(60) Provisional application No. 60/938,359, filed on May 16, 2007.
(60) Provisional application No. 60/545,576, filed on Feb. 17, 2004.
(60) Provisional application No. 60/566,818, filed on Apr. 30, 2004.
(60) Provisional application No. 60/400,624, filed on Aug. 2, 2002.
(60) Provisional application No. 60/423,881, filed on Nov. 5, 2002.
(60) Provisional application No. 60/423,881, filed on Nov. 5, 2002.
(60) Provisional application No. 60/400,624, filed on Aug. 2, 2002.

(51) Int. Cl. A61B 8/14

(52) U.S. Cl. 600/437

(57) ABSTRACT

A system includes at least one transducer configured to transmit at least one ultrasound pulse into a region of interest (ROI) of a patient. The pulse has at least a first frequency and propagates through a bodily structure in the ROI. The system further includes at least one receiver configured to receive at least one echo signal corresponding to the pulse. The echo signal includes the first frequency and at least one harmonic multiple of the first frequency. The system further includes a processor configured to automatically determine, from the at least one harmonic multiple, at least one boundary of the bodily structure. In an embodiment, the processor is configured to automatically determine, from the at least one harmonic multiple, an amount of fluid within the bodily structure.

### PRIORITY CLAIM AND RELATED APPLICATIONS

This application incorporates by reference and claims priority to U.S. provisional patent application Ser. No. 60/882,888 filed Dec. 29, 2006.

This application incorporates by reference and claims priority to U.S. provisional patent application Ser. No. 60/703,201 filed Jul. 28, 2005.

This application is a continuation-in-part of and claims priority to U.S. patent application Ser. No. 11/213,284 filed Aug. 26, 2005.

This application is a continuation-in-part of and claims priority to U.S. patent application Ser. No. 11/010,539 filed Dec. 13, 2004.

This application is a continuation-in-part of and claims priority to U.S. patent application Ser. No. 10/523,681 filed Feb. 3, 2005.

This application is a continuation-in-part of and claims priority to U.S. patent application Ser. No. 11/625,802 filed Jan. 22, 2007.

This application incorporates by reference and claims priority to U.S. provisional patent application Ser. No. 60/938,446 filed May 16, 2007.

This application incorporates by reference and claims priority to U.S. provisional patent application Ser. No. 60/938,359 filed May 16, 2007.

This application is a continuation-in-part of and claims priority to U.S. patent application Ser. No. 11/925,843 filed Oct. 27, 2007.

This application is a continuation-in-part of and claims priority to U.S. patent application Ser. No. 11/926,522 filed Oct. 27, 2007.

This application is a continuation-in-part of and claims priority to U.S. patent application Ser. No. 10/704,996 filed Nov. 10, 2003.

This application is a continuation-in-part of and claims priority to U.S. patent application Ser. No. 11/295,043 filed Dec. 6, 2005.

This application is a continuation-in-part of and claims priority to U.S. patent application Ser. No. 11/925,850 filed Oct. 27, 2007.

This application claims priority to and is a continuation-in-part of U.S. patent application Ser. No. 11/119,355 filed Apr. 29, 2005, which claims priority to U.S. provisional patent application Ser. No. 60/566,127 filed Apr. 30, 2004. This application also claims priority to and is a continuation-in-part of U.S. patent application Ser. No. 10/701,955 filed Nov. 5, 2003, which in turn claims priority to and is a continuation-in-part of U.S. patent application Ser. No. 10/443,126 filed May 20, 2003.

This application claims priority to and is a continuation-in-part of U.S. patent application Ser. No. 11/061,867 filed Feb. 17, 2005, which claims priority to U.S. provisional patent application Ser. No. 60/545,576 filed Feb. 17, 2004 and U.S. provisional patent application Ser. No. 60/566,818 filed Apr. 30, 2004.

This application is also a continuation-in-part of and claims priority to U.S. patent application Ser. No. 10/704,966 filed Nov. 10, 2004.

This application claims priority to and is a continuation-in-part of U.S. patent application Ser. No. 10/607,919 filed Jun. 27, 2005.

This application is a continuation-in-part of and claims priority to PCT application serial number PCT/US03/24368 filed Aug. 1, 2003, which claims priority to U.S. provisional patent application Ser. No. 60/423,881 filed Nov. 5, 2002 and U.S. provisional patent application Ser. No. 60/400,624 filed Aug. 2, 2002.

This application is also a continuation-in-part of and claims priority to PCT Application Serial No. PCT/US03/14785 filed May 9, 2003, which is a continuation of U.S. patent application Ser. No. 10/165,556 filed Jun. 7, 2002.

This application is also a continuation-in-part of and claims priority to U.S. patent application Ser. No. 10/888,735 filed Jul. 9, 2004.

This application is also a continuation-in-part of and claims priority to U.S. patent application Ser. No. 10/633,186 filed Jul. 31, 2003 which claims priority to U.S. provisional patent application Ser. No. 60/423,881 filed Nov. 5, 2002 and to U.S. patent application Ser. No. 10/443,126 filed May 20, 2003 which claims priority to U.S. provisional patent application Ser. No. 60/423,881 filed Nov. 5, 2002 and to U.S. provisional application 60/400,624 filed Aug. 2, 2002. All of the above applications are herein incorporated by reference in their entirety as if fully set forth herein.

### FIELD OF THE INVENTION

An embodiment of the invention relates generally to ultrasound-based diagnostic systems and procedures employing image acquisition, processing, and image presentation systems and methods.

### BACKGROUND OF THE INVENTION

Computer-based analysis of medical images pertaining to ascertaining organ structures allows for the diagnosis of organ diseases and function. Identifying and measuring organ boundaries allows a medical expert to assess disease states and prescribe therapeutic regimens. The true shape of a cavity or structure within body tissue requires accurate detection for the medical expert to assess organ normalcy or pathological condition. However, inaccurate organ boundary detection can prevent an accurate assessment of a true medical condition since the bladder cavity area and volume is either underestimated or overestimated. Traditional ultrasound technology employs the intensity information from the B-mode images for segmentation. However, due to the complex human anatomy and the artifacts of the ultrasound imaging, this B-mode information is insufficient. There is a need to non-invasively and rapidly identify and accurately measure cavity boundaries within an ultrasound-probed region-of-interest (ROI) so as to enable accurate assessment of a medical condition.

### SUMMARY OF THE PARTICULAR EMBODIMENTS

In an embodiment, a system includes at least one transducer configured to transmit at least one ultrasound pulse into a region of interest (ROI) of a patient. The pulse has at least a first frequency and propagates through a bodily structure in the ROI. The system further includes at least one receiver configured to receive at least one echo signal corresponding to the pulse. The echo signal includes the first frequency and at least one harmonic multiple of the first frequency. The system further includes a processor configured to automatically determine, from the at least one harmonic multiple, at least one boundary of the bodily structure. In an embodiment, the processor is configured to automatically determine, from the at least one harmonic multiple, an amount of fluid within the bodily structure.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee. Preferred and alternative embodiments of the present invention are described in detail below with reference to the following drawings.

FIGS. 1A-D depict a partial schematic and a partial isometric view of a transceiver, a scan cone comprising a rotational array of scan planes, and a scan plane of the array of an ultrasound harmonic imaging system;

FIG. 2 depicts a partial schematic and partial isometric and side view of a transceiver, and a scan cone array comprised of 3D-distributed scan lines in alternate embodiment of an ultrasound harmonic imaging system;

FIG. 3 is a schematic illustration of a server-accessed local area network in communication with a plurality of ultrasound harmonic imaging systems;

FIG. 4 is a schematic illustration of the Internet in communication with a plurality of ultrasound harmonic imaging systems;

FIG. 5 schematically depicts a progressive sound wave distortion with increasing harmonics;

FIG. 6 schematically depicts the super-positioning of fundamental, second and third harmonic wavelengths undergoing constructively interference;

FIG. 7A depicts a bladder image formed from multi-pulsed (50) ultrasound echoes at frequency 2.1 MHz, showing the overlap of ration of the second harmonic and the fundamental frequency component along scan lines within a bladder region of interest (ROI);

FIG. 7B depicts the frequency spectrum of the two sets of RF data in FIG. 7A;

FIG. 8 illustrates a process according to an embodiment of the invention;

FIG. 9A illustrates the harmonic information from 792 scan lines used for determining harmonic ratio profiles;

FIG. 9B is a schematic depiction of the harmonic echo response signal of a 3^{rd} harmonic ratio along scan lines at different theta angular values within a 2D scan plane;

FIG. 10 is a method to establish sufficient organ or structure aiming and to determine organ or structure boundary volume calculations using tissue harmonic images;

FIG. 11A is a color-coded presentation of a bladder in the pseudo Cmode view using the 3^{rd} ultrasound harmonic ratios on all scan lines from all 12 planes from FIG. 9B;

FIG. 11B is an interpolated shape in the pseudo C-mode view of the bladder based upon the segmentation;

FIG. 12 illustrates a process according to an embodiment of the invention;

FIG. 13A is a screenshot depiction of an aiming feedback of the not sufficiently targeted bladder;

FIG. 13B is a screenshot depiction of a virtual aiming aid of the aiming feedback presented in FIG. 13A;

FIG. 18 illustrates a harmonic analysis process according to an embodiment of the invention;

FIG. 19 illustrates a plot of the harmonic ratio vs. bladder size on each scan line from one human data set;

FIG. 20 illustrates a neural network employed by an embodiment of the invention;

FIG. 21 illustrates a process according to an embodiment of the invention;

FIG. 22 illustrates a projection of the bladder region according to an embodiment;

FIG. 23 illustrates a process according to an embodiment of the invention;

FIG. 24 illustrates arrow feedback modes according to an embodiment of the invention;

FIG. 25 illustrates rules for arrow-feedback display according to an embodiment of the invention;

FIG. 26 illustrates gradings for all lines in an exemplary data set according to an embodiment of the invention;

FIG. 27 illustrates a series of intermediate C-mode shapes on the exemplary data set according to an embodiment of the invention; and

FIG. 28 illustrates volume calculation according to an embodiment of the invention.

### DETAILED DESCRIPTION OF THE PREFERRED EMBODIMENTS

In at least one embodiment, ultrasound systems and methods employ harmonic theory to improve bladder segmentation. The reflected harmonic content associated with tissue regions beyond a volume of liquid, such as urine or amniotic fluid, to be measured is used to make a processing device, such as a computer, aware of the presence of the liquid. A color-coded image in pseudo C-mode view may be constructed based upon the strength of harmonic ratios from structures of a region-of-interest having structural components that increase the harmonic of the ultrasound waveform. The color-coded image can be utilized as a useful guidance for the task of aiming an ultrasound transceiver. In addition, harmonic ratio profile on each scan plane can be used to rectify the bladder (or uterus of non-pregnant female) region segmentation and fluid volume measurement.

In at least one embodiment, ultrasound systems and methods to develop, present, and use the harmonic theory which is only applied to voxels containing the harmonic information to improve bladder segmentation. A goal is to make assignment of regions preceding the bladder back wall tissue to the urine structure, instead of improving the image quality for visualization or image processing. Although there is little echo from a fluid such as urine or amniotic fluid, the propagation history through this type of liquid gives rise to additional decision making capability. The Goldberg number of the liquid is an advantageous indication in an embodiment. The harmonic content from bladder back wall tissue beyond the fluid are impacted by presence of the fluid in the ultrasound path in front of the tissue.

In at least one embodiment, ultrasound systems and methods develop, present, and use a color-coded image of a structure within a region-of-interest. A color-coded image of the structure or region-of-interest may be obtained based upon determining the optimal ultrasound harmonic frequency exhibited by the structure within the region-of-interest.

The harmonic distortion due to non-linear effects prevail in urine is an advantageous element of an embodiment. The concept of the harmonic is not new. For example, many methods have been proposed for using harmonic information to improve ultrasound image quality. In general, these methods use the reflected sound wave at all voxels and enhance the image quality at the corresponding location using its harmonic content. The harmonic information utilized in these applications is from all kinds of tissues. However, instead of improving the image quality for visualization or image processing, an embodiment models fluid in a bodily structure, such as urine inside a bladder, and tissue, such as bladder back wall and tissues behind it, as two different media for harmonic generation and absorption, so as to provide very useful information, such as the length of the path through the urine relative to the current scan path length.

The harmonic information is processed and utilized in a novel way. The entire propagation history information of each scan line is processed to provide a corresponding indicator. The urine in front of tissue will influence the harmonic information reflected from the tissue behind urine. Hence, regions composed of urine along the scan line contribute to the harmonic accumulation that appears on the structure behind the region. The urine itself is anechoic and generally does not present any image signal. Regions devoid of urine do not contribute to the harmonic accumulation. Without considering this accumulation process, looking at the harmonic information at each voxel independently will not provide information such as how much urine is presented in the current scan line. In short, we are not using harmonic image information; we are using harmonic propagation history information.

Another feature of an embodiment is the ultrasound propagation medium model employed. Instead of using harmonic information to differentiate different tissues as suggested by other approaches, we treat all the tissues with a single model. A focus may be the significant difference of harmonic propagations between tissue and urine, which is very clear from harmonic propagation theory. This treatment of the harmonic information gives us the opportunity to make fully or partially automatic determinations of how much urine is under examination, without human intervention based estimation of same.

In at least one embodiment, due to the above harmonic processing features, the transmitting signal we choose is narrowband, which is different from the wideband signals used for harmonic imaging. This is because we process the harmonic propagation history; hence the spatial resolution can be sacrificed and traded for better harmonic amplitude ratio estimation.

In at least one embodiment, for each imaging direction, an ultrasound transceiver transmits two pulses. The first one is a traditional B-mode pulse, while the second one is the narrowband pulse explained above for harmonic ratio estimation. The information obtained from the harmonic (second) pulse is merged with the B-mode information from the first pulse to provide a comprehensive view of the medium under examination. The successful fusion of these two pieces of information is another feature of an embodiment.

The quantitative harmonic amplitude estimation is a very challenging task due to the noisy nature of the spectrum and nonhomogeneous property of the signal. Many advanced spectral estimation algorithms have been developed in the literature to provide improved spectral estimation results for various engineering applications. Based on their principles, these algorithms can be divided into two approaches: parametric and nonparametric. Since the parametric approach is more sensitive to data modeling errors, the nonparametric approach are developed in an embodiment to build a robust spectral estimator. Careful studies of ultrasound propagation can lead to a good choice for this spectral estimator.

Other harmonic approaches use the absolute value of the second or higher harmonics as an indicator for volume rendering or threshold choosing. An embodiment uses the ratio between the second and the first harmonic to give us a better indicator, which is independent of the various echo generating capabilities of the tissues under examination. The conventional harmonic imaging approach cannot provide tissue harmonic absorbing information since the echo generating capability of the tissue will dominate the received signal.

There is a fundamental difference between an embodiment and a known alternative approach: an embodiment is concerned with the tissue harmonic absorption (this is why the harmonic propagation history of one scan line is processed herein), while the harmonic imaging technology from the alternative approach is concerned with the tissue harmonic generation. As discussed above in connection with our ultrasound propagation medium model, the model we selected for urine and tissue are based on their dramatically different harmonic absorption capabilities.

In at least one embodiment, systems and methods are described for acquiring, processing, and presenting a color-coded image in the pseudo C-mode view, based upon the strength of harmonic ratios from structures of a regions-of-interest having structural components that increase the harmonic of the ultrasound waveform. Optimization of image acquisition by providing systems and methods to direct transceiver placement or repositioning is described. When the structure or organ of interest, or region of interest (ROI) is a bladder, harmonic ratio classification results may be applied to alert the computer executable programs to check either or any combination of the volume measurement to properly determine a small or large bladder, the volume measurement of the bladder, and to adjust segmentation algorithms to prevent overestimation of the bladder size.

The result can also be combined with pseudo C-mode view displaying for transceiver aiming or final bladder shape determination. The simplest way to utilize the result may be that if the bladder size is large compared with harmonic ratio classification, we can check the dimension of current shape for over estimation. If the bladder size is too small, an appropriate compensation can be made to enlarge the size of the shape for displaying; if the size is small, we can provide an appropriate modification of the shape. In general, the harmonic ratio is an extra information extracted from the received ultrasound signal, which can be utilized to improve measurement of the bladder and/or fluid volume quantitatively.

Alternate embodiments include systems and/or methods of image processing for automatically segmenting (i.e., automatically detecting the boundaries of bodily structures within a region of interest (ROI) of a single or series of images undergoing dynamic change). Particular and alternate embodiments provide for the subsequent measurement of areas and/or volumes of the automatically segmentated shapes within the image ROI of a singular image or multiple images of an image series undergoing dynamic change.

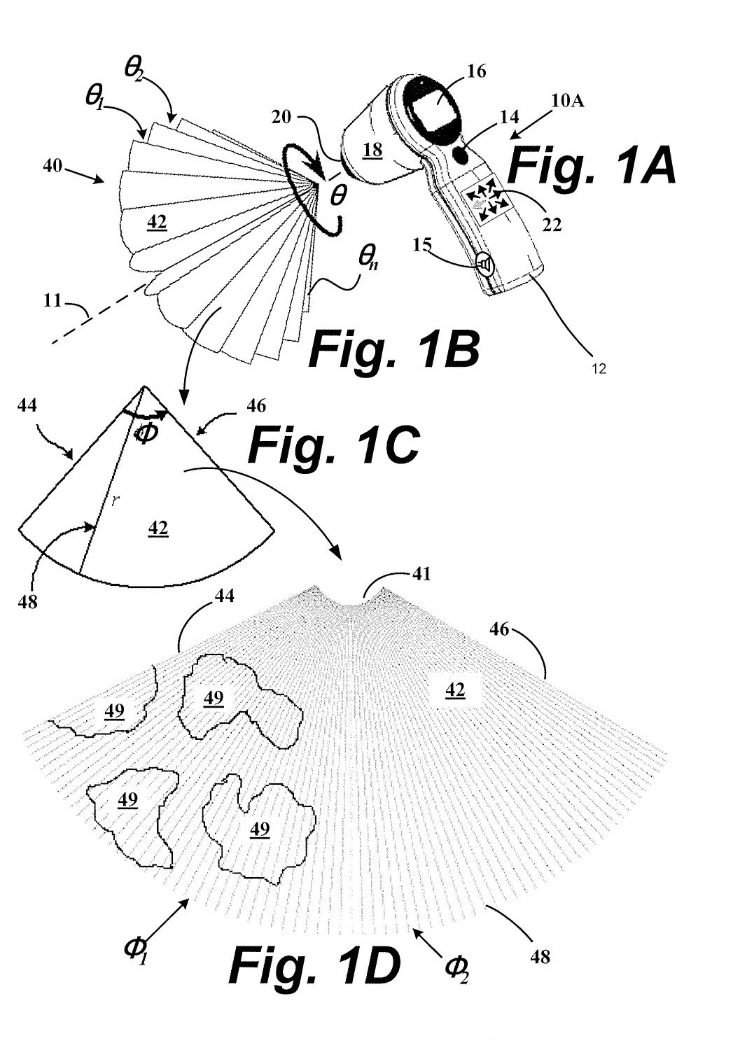

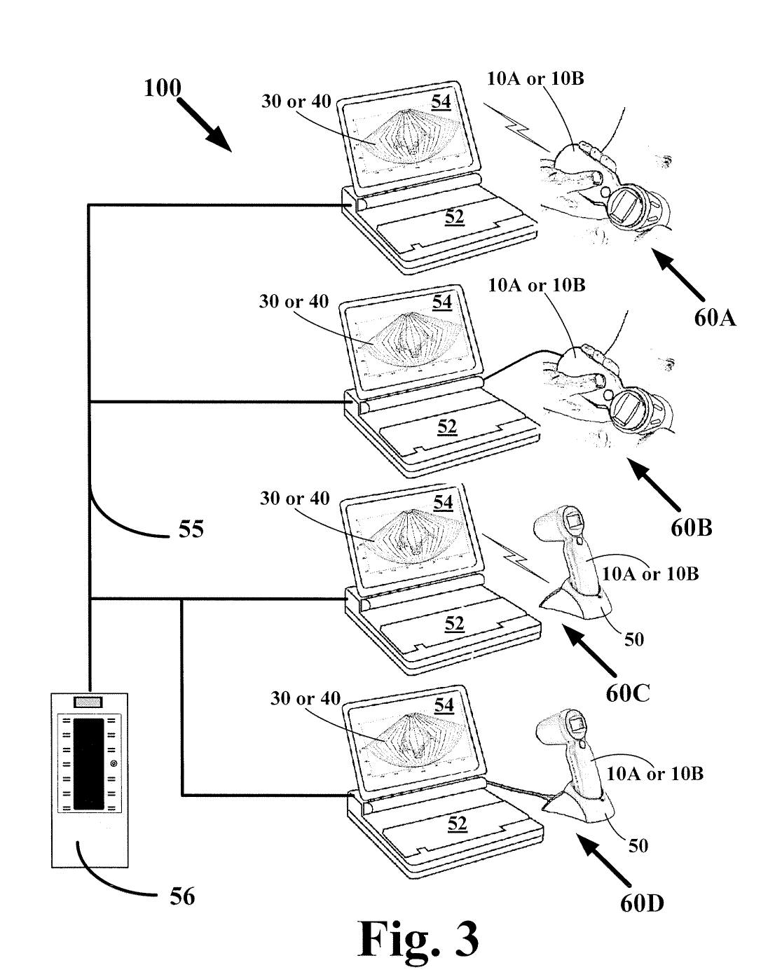

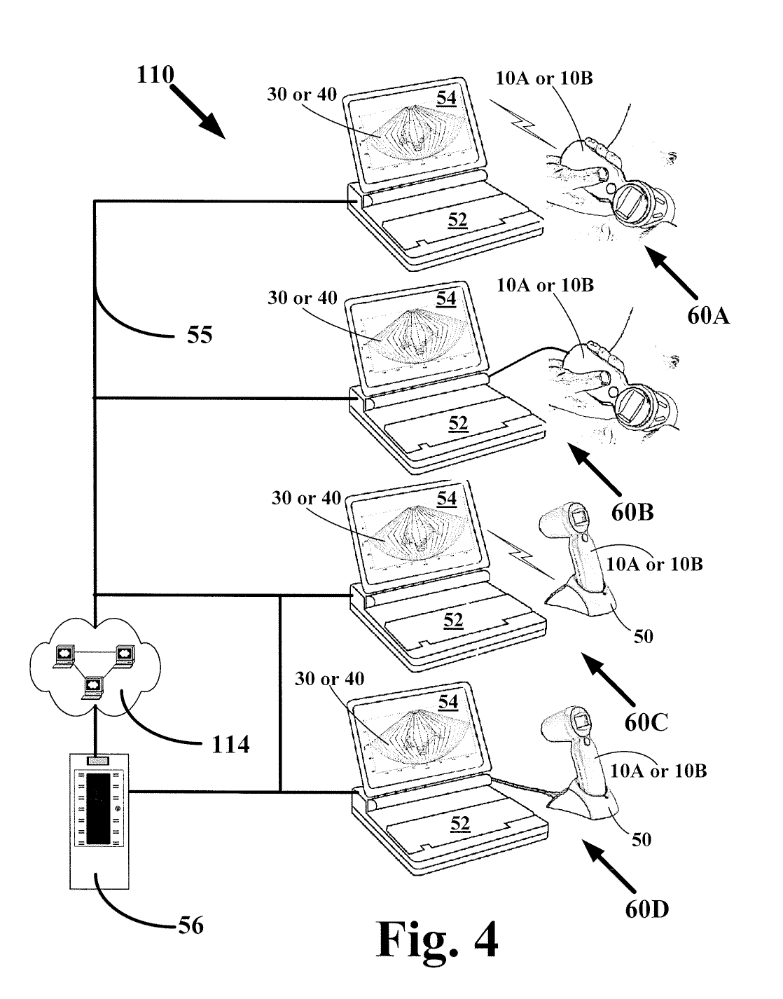

FIGS. 1A-D depicts a partial schematic and a partial isometric view of a transceiver, a scan cone comprising a rotational array of scan planes, and a scan plane of the array of various ultrasound harmonic imaging systems 60A-D illustrated in FIGS. 3 and 4 below.

FIG. 1A is a side elevation view of an ultrasound transceiver 10A that includes an inertial reference unit, according to an embodiment of the invention. The transceiver 10A includes a transceiver housing 18 having an outwardly extending handle 12 suitably configured to allow a user to manipulate the transceiver 10A relative to a patient. The handle 12 includes a trigger 14 that allows the user to initiate an ultrasound scan of a selected anatomical portion, and a cavity selector 16. The cavity selector 16 will be described in greater detail below. The transceiver 10A also includes a transceiver dome 20 that contacts a surface portion of the patient when the selected anatomical portion is scanned. The dome 20 generally provides an appropriate acoustical impedance match to the anatomical portion and/or permits ultrasound energy to be properly focused as it is projected into the anatomical portion. The transceiver 10A further includes one, or preferably an array of separately excitable ultrasound transducer elements (not shown in FIG. 1A) positioned within or otherwise adjacent with the housing 18. The transducer elements may be suitably positioned within the housing 18 or otherwise to project ultrasound energy outwardly from the dome 20, and to permit reception of acoustic reflections generated by internal structures within the anatomical portion. The one or more array of ultrasound elements may include a one-dimensional, or a two-dimensional array of piezoelectric elements that may be moved within the housing 18 by a motor. Alternately, the array may be stationary with respect to the housing 18 so that the selected anatomical region is scanned by selectively energizing the elements in the array.

A directional indicator panel 22 includes a plurality of arrows that may be illuminated for initial targeting and guiding a user to access the targeting of an organ or structure within an ROI. In particular embodiments if the organ or structure is centered from placement of the transceiver 10A acoustically placed against the dermal surface at a first location of the subject, the directional arrows may be not illuminated. If the organ is off-center, an arrow or set of arrows may be illuminated to direct the user to reposition the transceiver 10A acoustically at a second or subsequent dermal location of the subject. The acoustic coupling may be achieved by liquid sonic gel applied to the skin of the patient or by sonic gel pads to which the transceiver dome 20 is placed against. The directional indicator panel 22 may be presented on the display 54 of computer 52 in harmonic imaging subsystems described in FIGS. 3 and 4 below, or alternatively, presented on the transceiver display 16.

Transceiver 10A includes an inertial reference unit that includes an accelerometer and/or gyroscope (not shown) positioned preferably within or adjacent to housing 18. In case the ROI (region of interest) of one transceiver is not large enough to contain the organ of interest, such as when measuring the amniotic fluid, accelerometer and/or gyroscope can be used to merge several scans at different locations into one reference frame. The accelerometer may be operable to sense an acceleration of the transceiver 10A, preferably relative to a coordinate system, while the gyroscope may be operable to sense an angular velocity of the transceiver 10A relative to the same or another coordinate system. Accordingly, the gyroscope may be of conventional configuration that employs dynamic elements, or it may be an optoelectronic device, such as the known optical ring gyroscope. In one embodiment, the accelerometer and the gyroscope may include a commonly packaged and/or solid-state device. One suitable commonly packaged device is the MT6 miniature inertial measurement unit, available from Omni Instruments, Incorporated, although other suitable alternatives exist. In other embodiments, the accelerometer and/or the gyroscope may include commonly packaged micro-electromechanical system (MEMS) devices, which are commercially available from MEMSense, Incorporated. As described in greater detail below, the accelerometer and the gyroscope cooperatively permit the determination of positional and/or angular changes relative to a known position that is proximate to an anatomical region of interest in the patient.

The transceiver 10A includes (or if capable at being in signal communication with) a display (not shown) operable to view processed results from an ultrasound scan, and/or to allow an operational interaction between the user and the transceiver 10A. For example, the display may be configured to display alphanumeric data that indicates a proper and/or an optimal position of the transceiver 10A relative to the selected anatomical portion. Display may be used to view two- or three-dimensional images of the selected anatomical region. Accordingly, the display may be a liquid crystal display (LCD), a light emitting diode (LED) display, a cathode ray tube (CRT) display, or other suitable display devices operable to present alphanumeric data and/or graphical images to a user.

Still referring to FIG. 1A, a cavity selector 16 may be operable to adjustably adapt the transmission and reception of ultrasound signals to the anatomy of a selected patient. In particular, the cavity selector 16 adapts the transceiver 10A to accommodate various anatomical details of male and female patients. For example, when the cavity selector 16 is adjusted to accommodate a male patient, the transceiver 10A may be suitably configured to locate a single cavity, such as a urinary bladder in the male patient. In contrast, when the cavity selector 16 is adjusted to accommodate a female patient, the transceiver 10A may be configured to image an anatomical portion having multiple cavities, such as a bodily region that includes a bladder and a uterus. Alternate embodiments of the transceiver 10A may include a cavity selector 16 configured to select a single cavity scanning mode, or a multiple cavity-scanning mode that may be used with male and/or female patients. The cavity selector 16 may thus permit a single cavity region to be imaged, or a multiple cavity region, such as a region that includes a lung and a heart to be imaged.

To scan a selected anatomical portion of a patient, the transceiver dome 20 of the transceiver 10A may be positioned against a surface portion of a patient that is proximate to the anatomical portion to be scanned. The user actuates the transceiver 10A by depressing the trigger 14. In response, the transceiver 10 transmits ultrasound signals into the body, and receives corresponding return echo signals that may be at least partially processed by the transceiver 10A to generate an ultrasound image of the selected anatomical portion. In a particular embodiment, the transceiver 10A transmits ultrasound signals in a range that extends from approximately about two megahertz (MHz) to approximately about ten MHz.

In one embodiment, the transceiver 10A may be operably coupled to an ultrasound system that may be configured to generate ultrasound energy at a predetermined frequency and/or pulse repetition rate and to transfer the ultrasound energy to the transceiver 10A. The system also includes a processor that may be configured to process reflected ultrasound energy that is received by the transceiver 10A to produce an image of the scanned anatomical region. Accordingly, the system generally includes a viewing device, such as a cathode ray tube (CRT), a liquid crystal display (LCD), a plasma display device, or other similar display devices, that may be used to view the generated image. The system may also include one or more peripheral devices that cooperatively assist the processor to control the operation of the transceiver 10A, such a keyboard, a pointing device, or other similar devices. In still another particular embodiment, the transceiver 10A may be a self-contained device that includes a microprocessor positioned within the housing 18 and software associated with the microprocessor to operably control the transceiver 10A, and to process the reflected ultrasound energy to generate the ultrasound image. Accordingly, the display 24 may be used to display the generated image and/or to view other information associated with the operation of the transceiver 10A. For example, the information may include alphanumeric data that indicates a preferred position of the transceiver 10A prior to performing a series of scans. In yet another particular embodiment, the transceiver 10A may be operably coupled to a general-purpose computer, such as a laptop or a desktop computer that includes software that at least partially controls the operation of the transceiver 10A, and also includes software to process information transferred from the transceiver 10A, so that an image of the scanned anatomical region may be generated. The transceiver 10A may also be optionally equipped with electrical contacts to make communication with receiving cradles 50 as discussed in FIGS. 3 and 4 below. Although transceiver 10A of FIG. 1A may be used in any of the foregoing embodiments, other transceivers may also be used. For example, the transceiver may lack one or more features of the transceiver 10A. For example, a suitable transceiver need not be a manually portable device, and/or need not have a top-mounted display, and/or may selectively lack other features or exhibit further differences.

FIG. 1B is a graphical representation of a plurality of scan planes that form a three-dimensional (3D) array having a substantially conical shape. An ultrasound scan cone 40 formed by a rotational array of two-dimensional scan planes 42 projects outwardly from the dome 20 of the transceivers 10A. The other transceiver embodiments may also be configured to develop a scan cone 40 formed by a rotational array of two-dimensional scan planes 42. The pluralities of scan planes 40 may be oriented about an axis 11 extending through the transceivers 10A-10B. One or more, or preferably each of the scan planes 42 may be positioned about the axis 11, preferably, but not necessarily at a predetermined angular position θ. The scan planes 42 may be mutually spaced apart by angles θ₁ and θ₂. Correspondingly, the scan lines within each of the scan planes 42 may be spaced apart by angles φ₁ and φ₂. Although the angles θ₁ and θ₂ are depicted as approximately equal, it is understood that the angles θ₁ and θ₂ may have different values. Similarly, although the angles φ₁ and φ₂ are shown as approximately equal, the angles φ₁ and φ₂ may also have different angles. Other scan cone configurations are possible; for example, a wedge-shaped scan cone, or other similar shapes.

FIG. 1C is a graphical representation of a scan plane 42. The scan plane 42 includes the peripheral scan lines 44 and 46, and an internal scan line 48 having a length r that extends outwardly from the transceivers 10A-10B. Thus, a selected point along the peripheral scan lines 44 and 46 and the internal scan line 48 may be defined with reference to the distance r and angular coordinate values φ and θ. The length r preferably extends to approximately 18 to 20 centimeters (cm), although any length is possible. Particular embodiments include approximately seventy-seven scan lines 48 that extend outwardly from the dome 20, although any number of scan lines is possible.

FIG. 1D a graphical representation of a plurality of scan lines emanating from a hand-held ultrasound transceiver forming a single scan plane 42 extending through a cross-section of an internal bodily organ. The number and location of the internal scan lines emanating from the transceivers 10A-10B within a given scan plane 42 may thus be distributed at different positional coordinates about the axis line 11 as required to sufficiently visualize structures or images within the scan plane 42. As shown, four portions of an off-centered region-of-interest (ROI) are exhibited as irregular regions 49. Three portions may be viewable within the scan plane 42 in totality, and one is truncated by the peripheral scan line 44.

As described above, the angular movement of the transducer may be mechanically effected and/or it may be electronically or otherwise generated. In either case, the number of lines 48 and the length of the lines may vary, so that the tilt angle φ sweeps through angles approximately between −60° and +60° for a total arc of approximately 120°. In one particular embodiment, the transceiver 10 may be configured to generate approximately about seventy-seven scan lines between the first limiting scan line 44 and a second limiting scan line 46. In another particular embodiment, each of the scan lines has a length of approximately about 18 to 20 centimeters (cm). The angular separation between adjacent scan lines 48 (FIG. 1C) may be uniform or non-uniform. For example, and in another particular embodiment, the angular separation φ₁ and φ₂ (as shown in FIG. 1D) may be about 1.5°. Alternately, and in another particular embodiment, the angular separation φ₁ and φ₂ may be a sequence wherein adjacent angles may be ordered to include angles of 1.5°, 6.8°, 15.5°, 7.2°, and so on, where a 1.5° separation is between a first scan line and a second scan line, a 6.8° separation is between the second scan line and a third scan line, a 15.5° separation is between the third scan line and a fourth scan line, a 7.2° separation is between the fourth scan line and a fifth scan line, and so on. The angular separation between adjacent scan lines may also be a combination of uniform and non-uniform angular spacings, for example, a sequence of angles may be ordered to include 1.5°, 1.5°, 1.5°, 7.2°, 14.3°, 20.2°, 8.0°, 8.0°, 8.0°, 4.3°, 7.8°, and so on.

FIG. 1D is an isometric view of an ultrasound scan cone that projects outwardly from the transceivers of FIGS. 1-4. Three-dimensional images of a region of interest may be presented within a scan cone 40 that comprises a plurality of 2D images formed in an array of scan planes 42. A dome cutout 41 that is the complementary to the dome 20 of the transceivers 10A-10E is shown at the top of the scan cone 40.

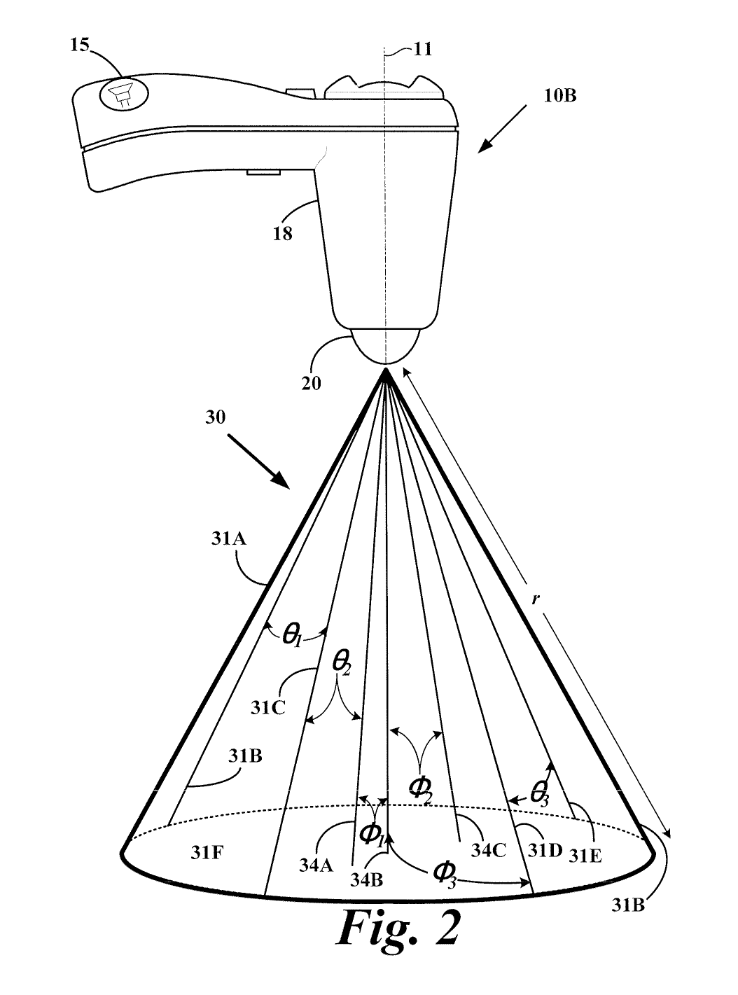

FIG. 2 depicts a partial schematic and partial isometric and side view of a transceiver, and a scan cone array comprised of 3D-distributed scan lines in alternate embodiment of an ultrasound harmonic ratio imaging system. A plurality of three-dimensional (3D) distributed scan lines emanating from a transceiver that cooperatively forms a scan cone 30. Each of the scan lines have a length r that projects outwardly from the transceivers 10A-10B. As illustrated the transceiver 10A emits 3D-distributed scan lines within the scan cone 30 that may be one-dimensional ultrasound A-lines. The other transceiver embodiment 10B may also be configured to emit 3D-distributed scan lines. Taken as an aggregate, these 3D-distributed A-lines define the conical shape of the scan cone 30. The ultrasound scan cone 30 extends outwardly from the dome 20 of the transceiver 10A, 10B centered about an axis line 11. The 3D-distributed scan lines of the scan cone 30 include a plurality of internal and peripheral scan lines that may be distributed within a volume defined by a perimeter of the scan cone 30. Accordingly, the peripheral scan lines 31A-31E define an outer surface of the scan cone 30, while the internal scan lines 34A-34C may be distributed between the respective peripheral scan lines 31A-31E. Scan line 34B is generally collinear with the axis 11, and the scan cone 30 is generally and coaxially centered on the axis line 11.

The locations of the internal and peripheral scan lines may be further defined by an angular spacing from the center scan line 34B and between internal and peripheral scan lines. The angular spacing between scan line 34B and peripheral or internal scan lines may be designated by angle Φ and angular spacings between internal or peripheral scan lines may be designated by angle Ø. The angles Φ₁, Φ₂, and Φ₃ respectively define the angular spacings from scan line 34B to scan lines 34A, 34C, and 31D. Similarly, angles Ø₁, Ø₂, and Ø₃ respectively define the angular spacings between scan line 31B and 31C, 31C and 34A, and 31D and 31E.

With continued reference to FIG. 2, the plurality of peripheral scan lines 31A-E and the plurality of internal scan lines 34A-D may be three dimensionally distributed A-lines (scan lines) that are not necessarily confined within a scan plane, but instead may sweep throughout the internal regions and along the periphery of the scan cone 30. Thus, a given point within the scan cone 30 may be identified by the coordinates r, Φ, and Ø whose values generally vary. The number and location of the internal scan lines emanating from the transceivers 10A-10B may thus be distributed within the scan cone 30 at different positional coordinates as required to sufficiently visualize structures or images within a region of interest (ROI) in a patient. The angular movement of the ultrasound transducer within the transceiver 10 may be mechanically effected, and/or it may be electronically generated. In any case, the number of lines and the length of the lines may be uniform or otherwise vary, so that angle Φ sweeps through angles approximately between −60° between scan line 34B and 31A, and +60° between scan line 34B and 31B. Thus angle Φ in this example presents a total arc of approximately 120°. In one embodiment, the transceiver 10A, 10B may be configured to generate a plurality of 3D-distributed scan lines within the scan cone 30 having a length r of approximately 18 to 20 centimeters (cm).

FIG. 3 is a schematic illustration of a server-accessed local area network in communication with a plurality of ultrasound harmonic imaging systems. An ultrasound harmonic imaging system 100 includes one or more personal computer devices 52 that may be coupled to a server 56 by a communications system 55. The devices 52 may be, in turn, coupled to one or more ultrasound transceivers 10A and/or 10B, for examples the ultrasound harmonic sub-systems 60A-60D. Ultrasound based images of organs or other regions of interest derived from either the signals of echoes from fundamental frequency ultrasound and/or harmonics thereof, may be shown within scan cone 30 or 40 presented on display 54. The server 56 may be operable to provide additional processing of ultrasound information, or it may be coupled to still other servers (not shown in FIG. 3) and devices. Transceivers 10A or 10B may be in wireless communication with computer 52 in sub-system 60A, in wired signal communication in sub-system 60B, in wireless communication with computer 52 via receiving cradle 50 in sub-system 60C, or in wired communication with computer 52 via receiving cradle 50 in sub-system 60D.

FIG. 4 is a schematic illustration of the Internet in communication with a plurality of ultrasound harmonic imaging systems. An Internet system 110 may be coupled or otherwise in communication with the ultrasound harmonic sub-systems 60A-60D.

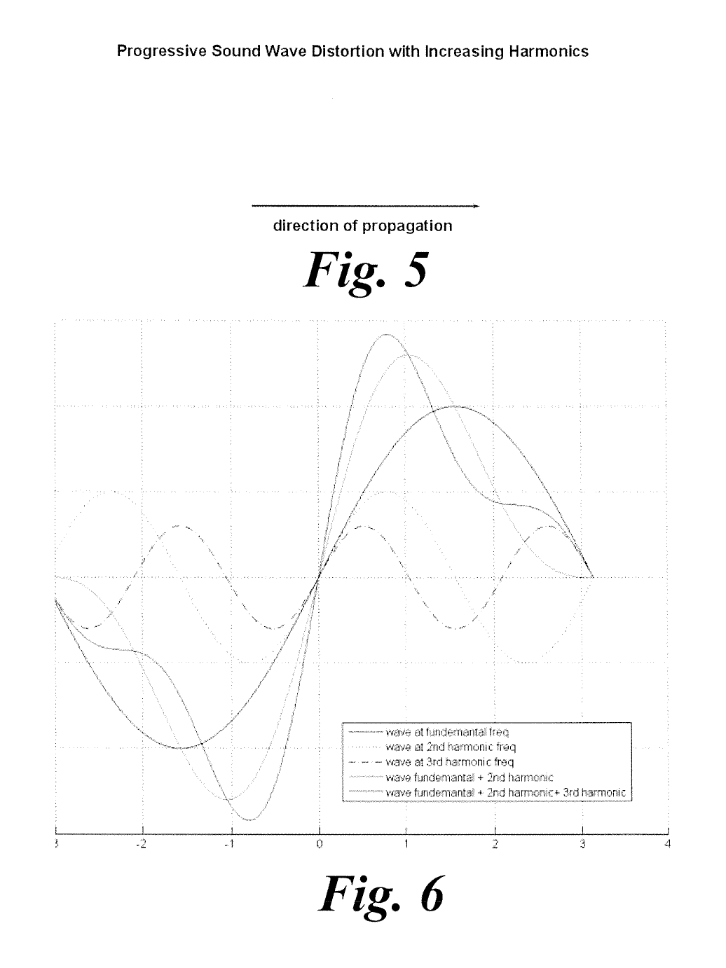

FIG. 5 schematically depicts a distortion of a waveform by propagation. Echo signals received from structures in the body carry not only the frequencies of the original transmit pulse, but also include multiples, or harmonics of these frequencies. Echoes from tissue have predominantly linear components, i.e. the echo frequencies are the same as the transmit frequencies. These linear components may be used in conventional, fundamental B-mode imaging. Non-linear effects cause harmonic echo frequencies during the propagation of ultrasound. Urine inside a bladder can greatly increase the harmonic components due to the low attenuation of harmonics in water.

One of the parameters that expresses the balance between attenuation and harmonic generation for an ultrasound wave is the Goldberg number, G, which represents a measure of the attenuation or harmonic distortion likely to prevail. When G=1, nonlinear effects become comparable to attenuation effect. If the Goldberg number is higher than 1, nonlinear processes dominate the wave propagation behavior. For values of the Goldberg number below 1, attenuation is more significant in governing the amplitude of the harmonic components than the energy transfer due to nonlinear distortion. Fat has a Goldberg number below 1 (0.27). Muscle, liver, and blood have a Goldberg number above but near 1. Urine and amniotic fluid have a Goldberg number of 104. This is caused primarily by the attenuation, which is very low for urine and very high for fat, although the nonlinearity coefficient of fat is higher than that of urine. These simple calculations demonstrate the difference between different media in causing waveform distortion. Urine and amniotic fluid have a higher ability to provoke strong nonlinear distortion compared with other body tissues. In an embodiment, the large Goldberg number value of urine and amniotic fluid is utilized to distinguish the bladder or umbilical region from other tissue regions.

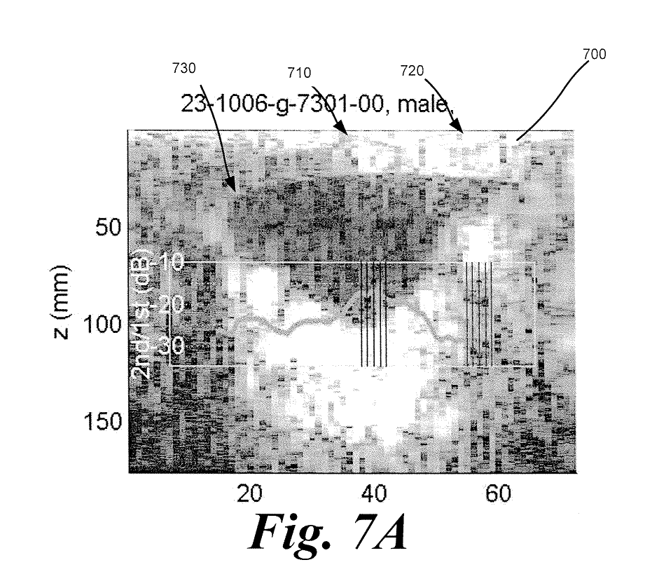

FIG. 6 schematically depicts the super-positioning of fundamental, second and third harmonic waveform undergoing constructive interference. The existing use of ultrasound harmonic frequencies to image structures is referred to as tissue harmonic imaging (THI) and is based on the effect that ultrasound signals are distorted while propagating through tissue with varying acoustic properties. The harmonic information utilized in these applications is from all kinds of tissues. THI provides an imaging application to better delineate structural boundaries of organs and cavities. However, as discussed in the previous sections, the harmonic information used in an embodiment is different from such a conventional approach. The harmonic distortion due to non-linear effects associated with a fluid, such as urine, normally invisible to a conventional harmonic imager due to its anechoic nature, is an optionally advantageous feature of an embodiment. The harmonic information utilized in an embodiment is not from all kinds of tissues. In at least one embodiment, a method is to model the urine inside the bladder and tissue as two different media for harmonic generation and absorption, so we can provide very useful information, such as if the ultrasound wave is passing a urine-filled region and relatively how much urine is in the current ultrasound scan path. It is the propagation history information through the urine in front of the tissue giving rise to a decision-making capability. Color-coded legends for the fundamental, second harmonic, third harmonic, super positioning of the fundamental and second harmonic, and super-positioning of the fundamental, second, and third harmonics are presented on the figure.

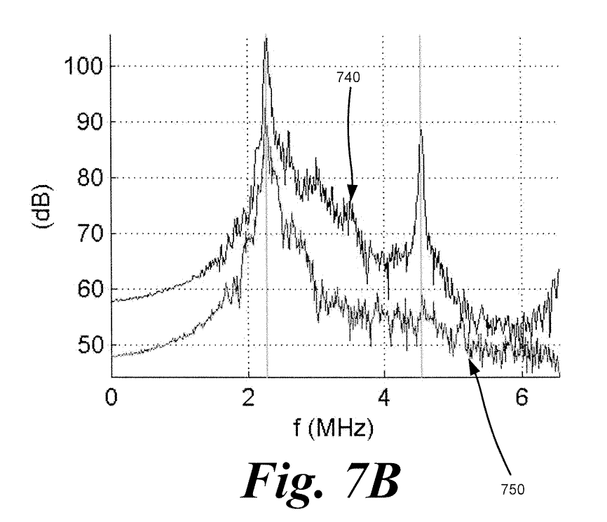

FIG. 7A depicts graphical results of a test on a human subject. The test included 50 pulses of ultrasound wave at frequency on 2.1 MHz and we only collected the RF signals at the depth range inside the yellow window 700. A b-mode image is formed using the received RF data. As such, FIG. 7A depicts a bladder image formed from multi-pulsed (50) ultrasound echoes at frequency 2.1 MHz, showing the overlap of harmonic ratio (the second harmonic over the fundamental frequency component) along scan lines within a bladder region of interest (ROI). Using the five blue RF lines 710, we computed the maximum ratio value. Using the five red RF lines 720, we computed the minimum ratio value. From this example, we can see the harmonic response is lower at the scan lines which are not passing a bladder region filled with urine, than at the scan lines which are passing through bladder region. We compared the frequency responses at two scan lines, blue and red. We found that the intensity of the response around the 2^{nd} harmonic is very different from the red to the blue line. We define the ratio of the average value around the 2^{nd} harmonic and the average value around the fundamental frequency as an indicator of the measurement. In the figure, the green curve 730 represents this measurement on all scan lines inside the yellow window 700.

FIG. 7B depicts the frequency spectrum of the two sets of RF data in FIG. 7A. Colors in FIG. 7C correspond to colors of regions in FIG. 7A (i.e., 740 corresponds to 710; 750 corresponds to 720). The blue RF 740 has a larger second harmonic component in the frequency domain than the red RF 750.

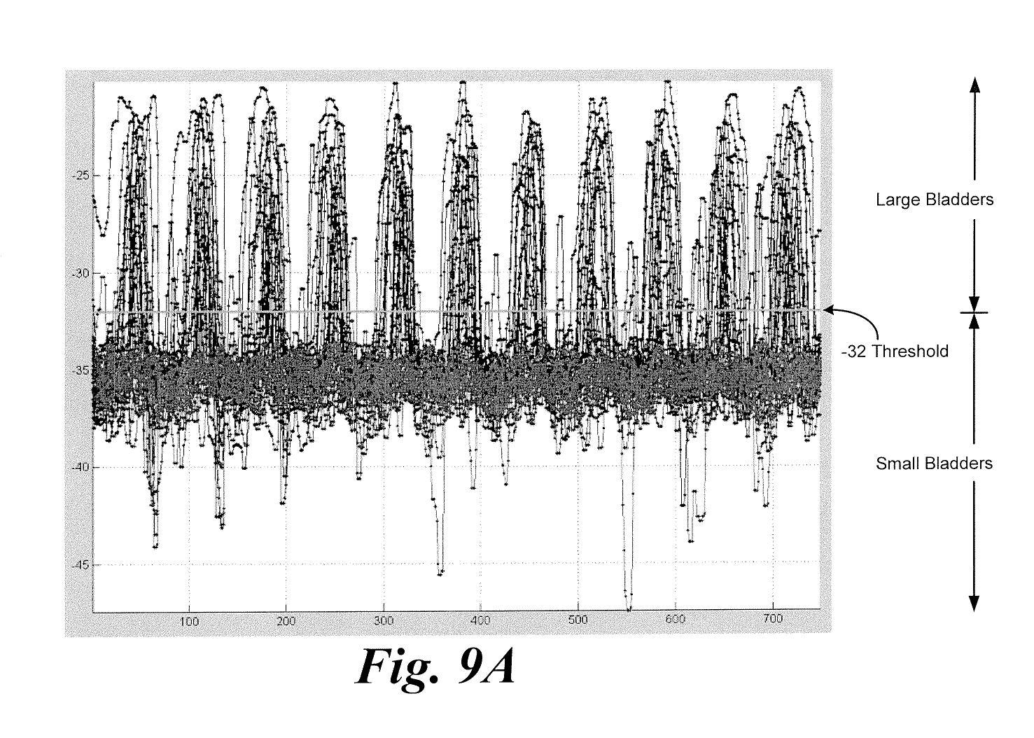

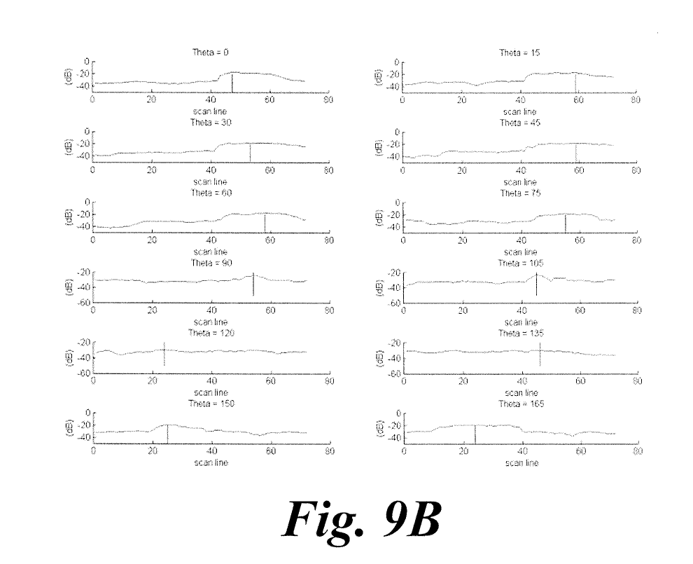

FIG. 9A illustrates an 11-scan plane sampling of 12 scan planes used for determining harmonic ratio profiles. Each scan plane is derived from 72 scan lines. The harmonic ratios may be determined from 12 data sets derived from the 12 scan planes. A threshold of approximately −32 dB may be defined to be the harmonic ratio used to classify or generally demarcate a small bladder from a large bladder. The blue data sets are the harmonic ratios along each scan line on the 11 planes. The corresponding data was collected from a human subject with large bladder volume. The red data sets are the harmonic ratios along each scan line on the 11 planes. The corresponding data was collected from a human subject with small bladder volume.

FIG. 9B is a schematic depiction of the harmonic ratio along scan lines at different theta angular values within twelve 2D scan planes. Harmonic information of the 12 scan planes for a single scan cone may be interpolated based on these 12 profiles corresponding to theta angular values of 0, 15, 30, 45, 60, 90, 105, 120, 135, 150, and 165 degrees. The settings employed use a fundamental frequency of 2.46 MHz with a pulse number as 20.

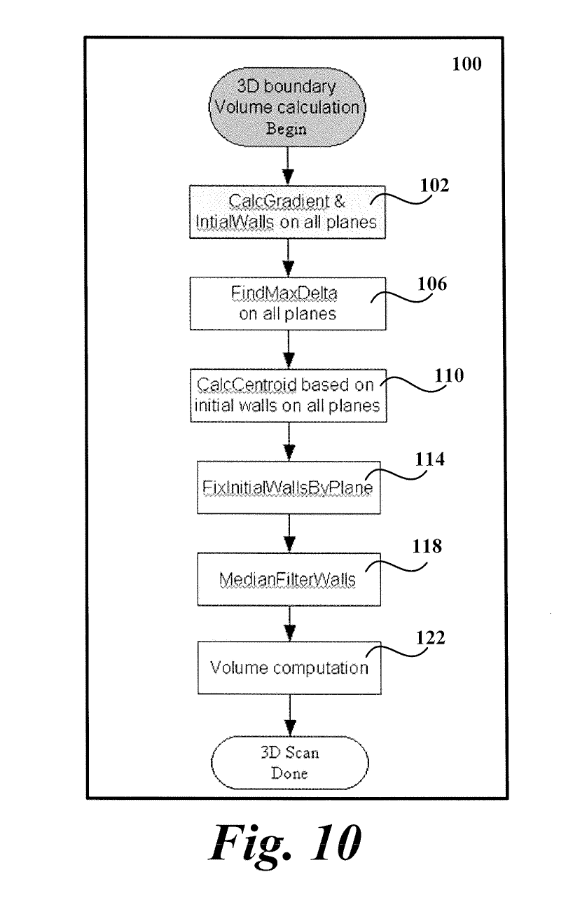

FIG. 10 is a method to establish sufficient organ or structure aiming and to determine organ or structure boundary volume calculations using harmonic ratios. When the organ is a bladder, a bladder volume instrument (BVI) aiming and segmentation method begins by using a harmonic ratio peak is for initial wall localization at process block 102 wherein the boundary volume calculation method 100 utilizes the Calculate-Gradient and Initial-Walls on the scan planes. The gradient information corresponds to each scan line. Since the approach is based on hard thresholds, inevitably, it will lead to some non-ideal initial wall candidates. The regions above the harmonic ratio threshold can be taken as another set of bladder wall candidates. Or the harmonic ratio can be taken as extra criterion for initial wall candidates selection. Thereafter, at process block 106, a Find-Max-Delta is determined, followed by calculating the centroid based on the initial walls on all planes at process block 110. The harmonic ratio peak is for wall fixing and is based on the centroid being modified to determine the location with MaxDelta on each plane so that the peak location of the harmonic ratio provides extra information to determine if the starting location for wall fixing is appropriate. Thereafter, at process block 114, a Fix-Initial-Walls-By-Plane process is accomplished, followed by application of a Median-Filter-Walls at process block 118. The method 100 is then finished by completing process block 122 Volume computation to determine the volume of harmonic imaged and segmented structures within a region-of-interest. The image and data processing algorithms, including polynomial differential formulas (PDF) that delineate the bladder front and/or back walls of the method 100 may be adapted from the VTK Library maintained by Kitware, Inc. (Clifton Park, N.Y., USA), incorporated by reference herein.

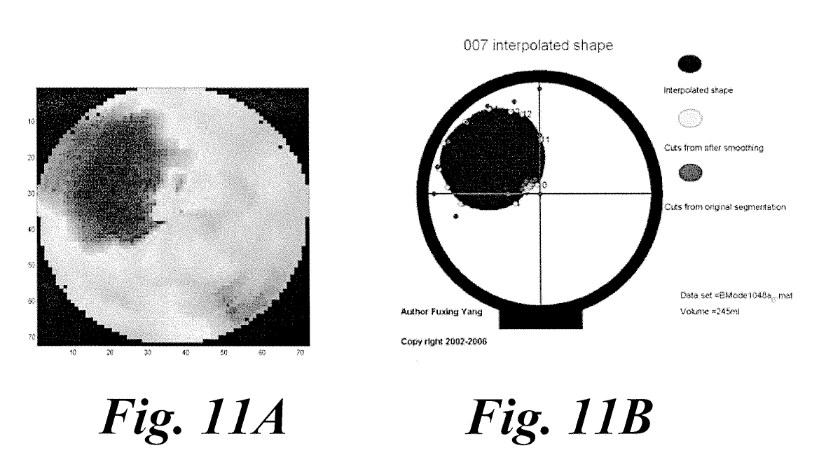

FIG. 11A is a color-coded presentation of a bladder in the pseudo C-mode view using the 3^{rd} ultrasound harmonic ratios on all scan lines from all 12 planes from FIG. 9B. The color-coded image may be obtained from a 32 bit jet color map. The red color represents a bladder region, while the blue represents a non-bladder region.

FIG. 11B is an interpolated shape in the pseudo C-mode view of the bladder based upon the segmentation. It can be found that there is very close correspondence between the bladder region based on segmentation and the red region from the harmonic ratio in FIG. 11A. FIG. 11B is an interpolated shape of the bladder based upon the color-coded presentation of FIG. 11A. The red color in FIG. 11A represents the bladder region, while the other colors represent the non-bladder region. For this result, the bladder shape based on the link of the segmentation from all planes is shown and has close correspondence with the harmonic ratio image of FIG. 11A. This new imaging method can be utilized as a very useful guidance for the task of aiming the transceiver.

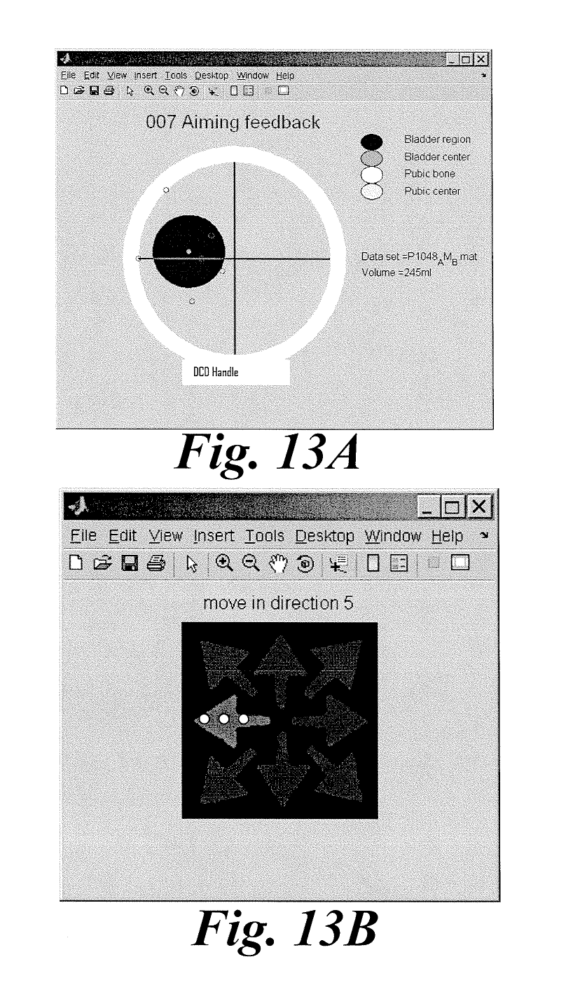

FIG. 13A is a screenshot depiction of an aiming feedback of the not ideally targeted bladder. The cross hairs of the targeting images may be beyond the segmented boundary of the blue bladder region.

FIG. 13B is a screenshot depiction of a virtual aiming aid of the aiming feedback presented in FIG. 13A. Since the cross hairs of the targeting image is outside of the blue bladder region, a leftward arrow with three circles is illuminated to indicates the direction of movement of that the transceiver 10A or 10B to be undertaken to obtain a centered bladder image. Here the statement “move in direction 5” is shown above the virtual aiming aid.

The color-coded images using harmonic ratio specially designed for bladder aiming/targeting is employed in an embodiment. The method is based on the special property of the non-linear propagation of ultrasound wave. A fast interpolation and efficient color map may be explored and a 2D pseudo-color imaging can be generated for each bladder scan. Operator can easily find the urine-filled bladder in the image and adjust scanning direction for best aiming.

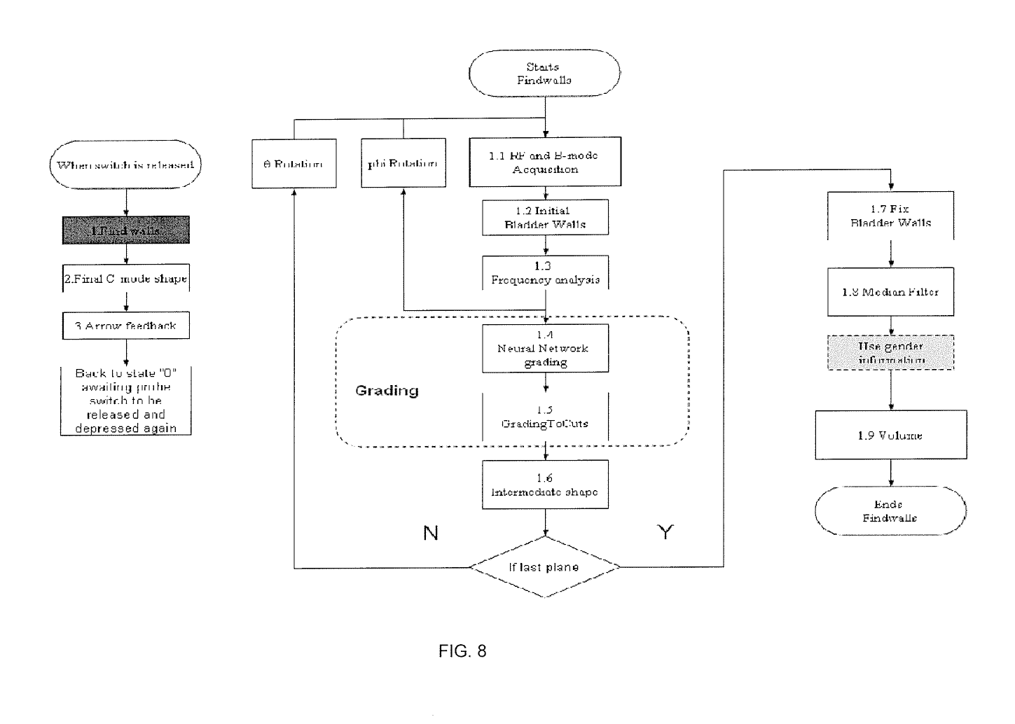

Based on the initial design and implementation of 30 tests on human subjects, and using the interpolated shape as reference, the color harmonic imaging method provides accurate feedback about, for example, bladder location, bladder shape and bladder volume. This technique can be easily applied for clinical usage for more accurate data collection and analysis. A process according to an embodiment is illustrated in FIG. 8. In step 1.1, on each plane, a transceiver collects two RF signals for B-mode imaging and harmonic content extraction. Initial walls are estimated at step 1.2. Step 1.3 is the harmonic analysis kernel, as explained in greater detail below herein. At step 1.4, an embodiment employs a pre-trained neural network (described in greater detail below herein) to give grading for each line on a current plane using the harmonic ratio information and other related features based on intensity information. The higher the grade is, the larger the possibility that the scan line is through the bladder region with urine. The grading is utilized to fix the segmentation at step 1.5. The fixed segmentation will be used for bladder volume measurement at step 1.9. More details of the steps are given in the following sections.

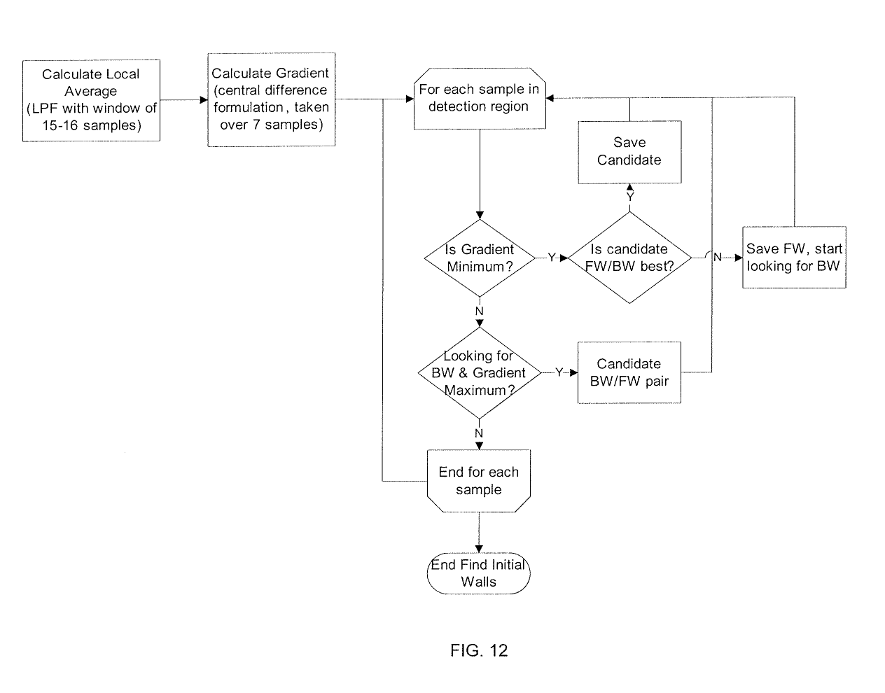

FIG. 12 illustrates the initial bladder wall detection process (Step 1.2 illustrated in FIG. 8) according to an embodiment. This process may be executed on every A-mode scan line. The first step here is local averaging/low-pass filtering using a 15 or 16 sample window. Next, a local gradient is computed for each sample point using a central difference formulation. Next, for each scan line, the algorithm tries to find the best front wall (FW) and back wall (BW) pair. The best front wall and back wall pair on each line is defined as the front wall and back wall pair for which the difference in the back wall gradient and front wall gradient (also called the tissue delta) is the maximum and the local average between front wall and back wall pair is the minimum.

At step 1.3 illustrated in FIG. 8, harmonic frequency analysis is performed. In general, prior approaches were designed to extract the scan lines that pass the bladder region, based on B-mode image. However, artifacts such as reverberations and shadows degrade ultrasound images. Therefore, the corresponding gradient information in B-mode images may be incomplete for these cases and lead to erroneous bladder detection.

Echo signals received from structures in the body carry not only the frequencies of the original transmit pulse, but also include multiples, or harmonics of these frequencies. These linear components are used in conventional, fundamental B-mode imaging. Harmonic echo frequencies are caused by non-linear effects during the propagation of ultrasound.

For example, THI (tissue harmonic imaging) is based on the phenomenon wherein ultrasound signals are distorted while propagating through tissue with varying acoustic properties. However, THI is merely an imaging method that does not solve the bladder detection problem.

Harmonic information is hidden in the frequency domain and it is an effective indicator for harmonic build-up on each scan line at different depth, based on which bladder lines and tissue lines can be separated. For example, inside a bladder region, there is not enough reflection, so the attenuations of the first and second harmonics are low. Deep behind the bladder wall, both the first and the second harmonics will be attenuated, while the second harmonic will be attenuated much faster than the first one. As a result, harmonic information will be higher for a scan line which passes through a bladder, compared to a scan line that penetrates tissue only.

One way to use the harmonic information is to use relative change of the harmonic information around the 2nd harmonic frequency compared with response at fundamental frequency. The ratio (Goldberg Number) of the peak value around the 2nd harmonic and the peak value around the fundamental frequency is a suitable indicator for such change.

From the clinical data collected from an ultrasound device, it can be observed that its spectrum is very noisy. This holds true even when there is little or no noise presented within the data. The convolution theory indicates that it is hard to use conventional FFT method to get good spectral estimation, not to mention that the stationary assumption does not hold for this data. A robust harmonic processing algorithm enables such a device to have good harmonic estimation results.

Many advanced spectral estimation algorithms have been developed in the literature to provide improved spectral estimation results for various applications. Based on their principle, these algorithms can be divided into two approaches: parametric and nonparametric. Since the parametric approach is more sensitive to data modeling errors, an embodiment includes the nonparametric approach to build a robust spectral estimator.

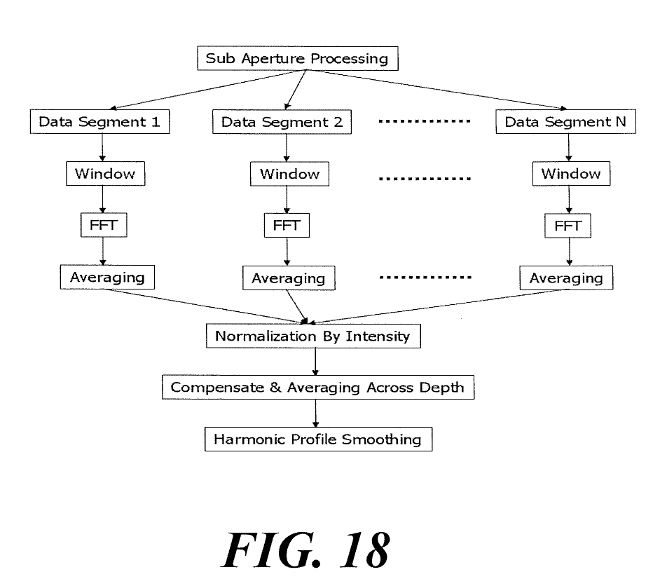

A block diagram of the Harmonic Analysis Kernel is illustrated in FIG. 18. In general, such an approach may be based on sub-aperture processing technology, and it can be approximately regarded as a deconvolution process. The sub-aperture processing technology is ideal, in an embodiment, since it can be approximately regarded as a deconvolution process. The resulting data segments can be either overlapping or non-overlapping. For each data segment (on a single RF data line), a Taylor window is applied to reduce its sidelobes from FFT. After FFT, we average its spectrum around the first and the second harmonic frequencies. Next, we ‘normalize’, compensate, and average the harmonic ratios based on the following sub-algorithm:

```
      Ratio_Sum = 0;
      Counter = 0;
    For each data segment(i)
        If (first harmonic>threshold)
          Ratio_SA(i) = 20*log10(first harmonic/second
harmonic);
          Ratio_SA(i) = Ratio_SA(i) + i*Att_Comp;
          Ratio_Sum = Ratio_Sum + Ratio_SA(i);
          Counter = Counter +1;
        End if
      End for
      If (Counter > 0)
        Ratio = Ratio_Sum/Counter;
      Else
        Ratio = Ratio_low
      End if
```

In the above sub-algorithm, ‘Att_Comp’ is an attenuation compensation parameter (we use 2.5 dB/cm, estimated from clinical data). The ‘threshold’ is a parameter used to reject the data when they are too small. Ratio_low=−35 dB. In summary, the ‘normalization’ step will remove the data segments which are too weak, the compensation step will compensate the harmonic ratio loss in tissue, and the averaging step will provide a more robust ratio estimator.

The final step may be spatially smoothing the harmonic ratios across the scan lines within a plane.

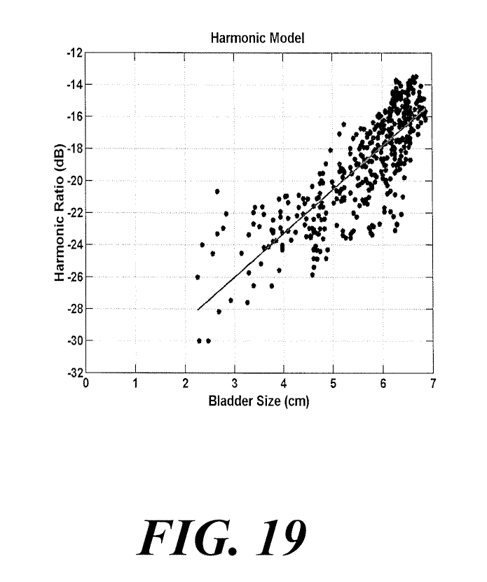

Data collected from a clinical test has been used to validate the model: more urine will lead to more harmonic. FIG. 19 illustrates a plot of the harmonic ratio vs. bladder size on each scan line from one human data set. Each blue point indicates the harmonic ratio corresponding to a scan line through a bladder. Clearly, there is a linear relationship between bladder size and the corresponding harmonic ratio. If we fit the data into a linear model, which is indicated by the red line, it has a slope of 2.726 dB/cm. This result matches the theoretical value well. The intersection between the linear model and the y-axis may be our baseline for this image:harmonic ratio with no bladder presented. This would be −34 dB according to the plot of FIG. 19.

Previous bladder detection methods are focused only around the gradient information from B-mode images. As discussed elsewhere herein, artifacts in ultrasound images create difficulties. Harmonic information provides extra features from the frequency domain and the combination improves application accuracy.

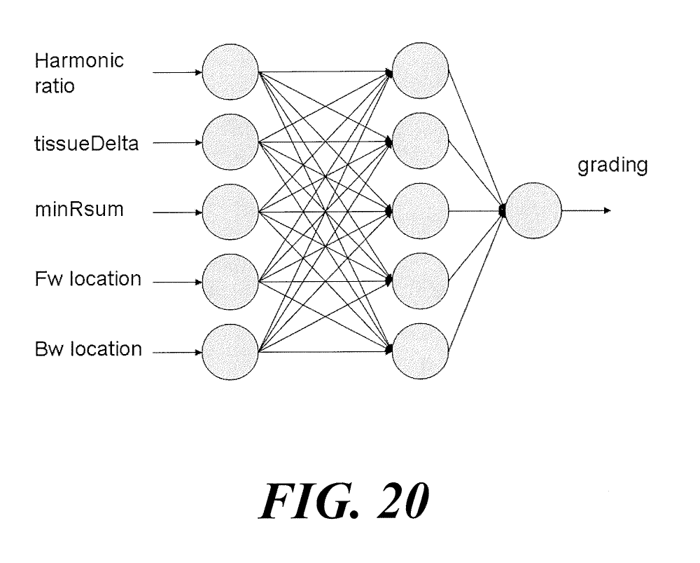

An embodiment includes combining harmonic features with B-mode image properties. Such an approach may include a pre-trained 5 by 5 by 1 Neural Network [FIG. 20], with different features as inputs and a single grading [0-1] as output. For each scan line, after initial walls are estimated based on gradient information, the corresponding features will be computed and the grading value from this network will show how likely it is that the current line is a bladder line.
If the grading is low, that means the current line is very likely a tissue line. The initial walls may be wrong or there should be no walls at all.
If the grading is high, that means the current line is very likely a bladder line. The initial walls may be correct.

The neural network may require exponential calculation in a logistic function [logistic(x)=1.0/(1+exp(−x))]. In the DSP processing, an embodiment uses a lookup table to give a fast implementation.

The trained network may be in the following configuration:

| | |
|---|---|
| #define n_input_units | 5 |
| #define n_hidden_units | 5 |
| #define n_output_units | 1 |
| #define na_input_units | n_input_units + 1 |
| #define na_hidden_units | n_hidden_units + 1 |
| #define na_output_units | n_output_units + 1 |

const double BPN N_IH[na_input_units][na_hidden_units] =
{

| | | | | | |
|---|---|---|---|---|---|
| {0, | 0, | 0, | | 0, 0, | 0}, |
| {0, | 13.008636, | −5.242537, | −8.093809, | 0.738920, | −1.345708}, |
| {0, | 2.039624, | 2.109022, | −3.339866, | −3.926513, | −6.129284}, |
| {0, | −4.525894, | −4.832823, | 3.689193, | −3.612824, | −1.418404}, |
| {0, | −6.834694, | −3.932294, | 7.301636, | 0.151018, | −6.567073}, |
| {0, | −0.997530, | −6.582561, | 1.040930, | −4.179786, | 6.771766 } |

```
};
const double BPN N_HO[na_hidden_units][2] =
{
    {0,0},
    {0,2.654482},
    {0,−17.31553},
    {0,−1.429942},
    {0,−11.77292},
    {0,−2.519807}
};
const double maxfeature[na_input_units] =
{0, 238.7272727, 43.49219326, 2048, 294, 536 };
const double min feature[na_input_units] =
{0, 0, 6.46712798, 1, 0, 0};
```

An embodiment may use harmonic information for bladder detection (Grading on Walls). The goal of using harmonic information is to improve liquid-volume measurement accuracy and help a user locate a bladder region faster. The goal is directly related to the segmentation accuracy of the bladder region. With the harmonic information, we can check if the segmentation (detection of bladder walls) on each scan line is valid. The grading from the neural network provides more robust information to fix the initial bladder walls.

The basic idea is to use the grading value:
to remove the bladder walls with too small grading; and
to add new bladder walls with large grading using the nearest valid initial bladder wall pair.

In an embodiment, a region G is defined in which all lines are with grading higher than the threshold. Additionally, a region W is defined which is based on the cuts from fixed walls.

For Region G and region W, there may be five different cases to address:

(1) G and W are not Overlapped (Including Empty G or Empty W):

| Action: | remove both |
|---|---|
| G | \|----------\| |
| W | \|----------\| |

(2) G Inside W:

| Action: | remove the walls in W, that are not in G |
|---|---|
| G | \|-------------------\| |
| W | \|------------------------------\| |

(3) W Inside G:

| Action: | add the walls outside W, that are in G |
|---|---|
| G | \|------------------------------\| |
| W | \|-------------------\| |

(4) G and W are Partly Overlapped:

| | remove the walls in W, that are not in G, and add |
|---|---|
| Action: | the walls outside W, that are in G |
| G | \|-----------------\| |
| W | \|-------------------\| |

(5) G and W are Exactly the Same:

| Action: | none |
|---|---|
| G | \|-----------------\| |
| W | \|-----------------\| |

It is easy to remove the wrong segmentation line. But, it is difficult to add new lines. An embodiment determines the average of the non-zero initial wall on current line and the non-zero fixed wall from its neighbor.

The bladder detection task is more challenging for a female patient due to the presence therein of a uterus. In general, the uterus is adjacent to the bladder region and it has a very similar pattern in B-mode image.

It is optionally advantageous to exclude the uterus region from the final segmentation. Therefore, the computed volume is the actual urine inside the bladder. Previously, a uterus detection method may address the whole segmentation after wall detection using volume. In other words, it tries to determine that the segmentation is bladder or uterus. However, some times, it is not so simple to refine the result, because the segmentation includes both bladder and uterus. An embodiment may determine which part in the segmentation belongs to the bladder and which part in the segmentation is the uterus. This may be a difficult task, especially when the bladder is small in size.

The uterus can be located side by side with the bladder, and it can also be located under the bladder. For the first case, a method previously described herein can be used to classify the scan lines passing through uterus only from the scan lines passing through bladder. However, such a method may not be able to solve the second problem. When a scan line is propagating through both bladder region and uterus region, further processing has to be made to find which part on the line belongs to the bladder.

An embodiment is based on the following observation: if the scan is on a female patient, there must be a boundary between uterus and bladder region and the uterus is always under the bladder if both regions appear on a scan line. In the B-mode image, for each scan line passing through both regions, a small ridge exists. If the ridge can be located, an embodiment can tell the two structures apart.

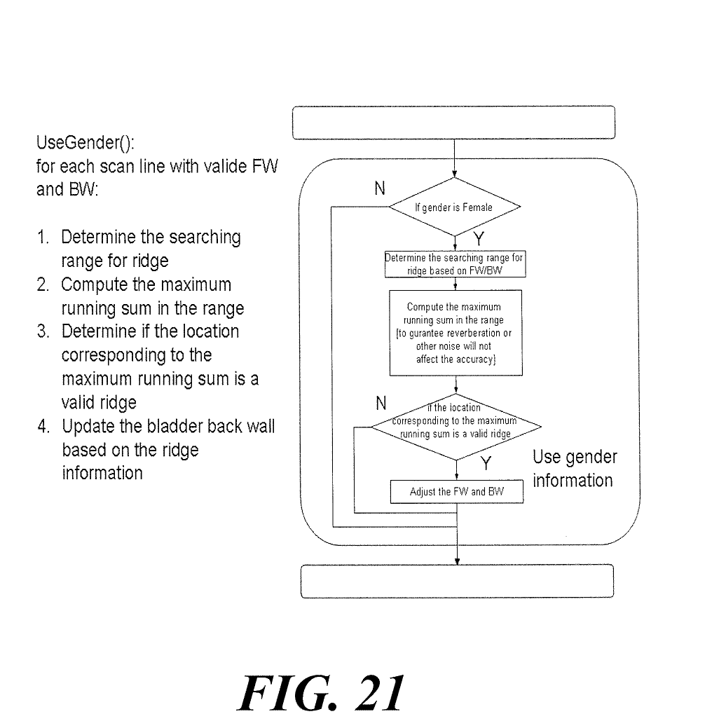

A detailed design of an embodiment of this procedure is illustrated in FIG. 21.

Referring again to FIG. 8, at step 2, for human operators, an embodiment provides the function called C-mode shape displaying. The goal of this functionality is to show the location and size information of the bladder or other structure in a current scan, based on which, users are able to adjust scan direction and angle. The shape is generated based on the segmentation on all scan lines.

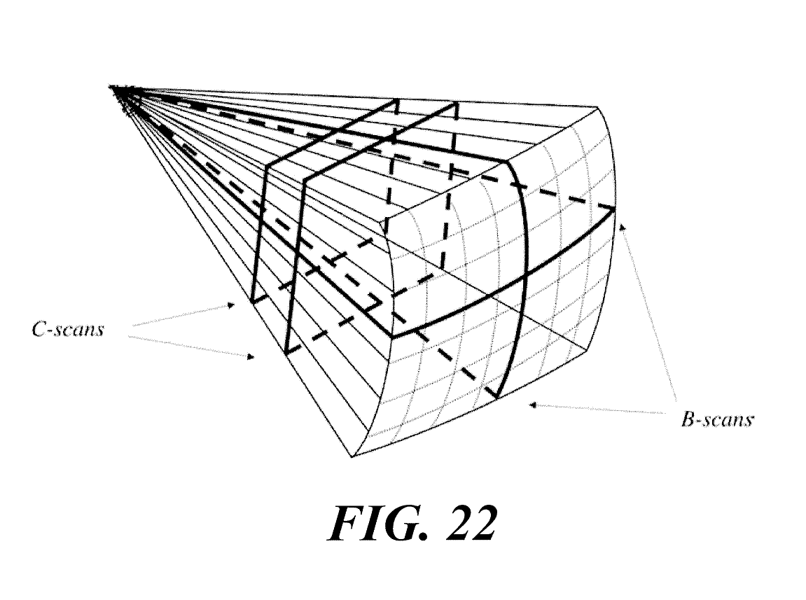

The definition of a C-mode image may be a plane parallel to the face of the transducer. As illustrated in FIG. 22, an embodiment provides to the users the projection of the bladder region. Consequently, the information is not only from a single plane parallel to the transducer surface. As such, it may be called a pseudo C-mode image. In an embodiment, the image is binary, including non-bladder region and bladder region. The bladder region [a.k.a. Interpolated shape] may be generated from the left most and the right most cuts on all planes. [cut: valid segmentation of bladder region.]

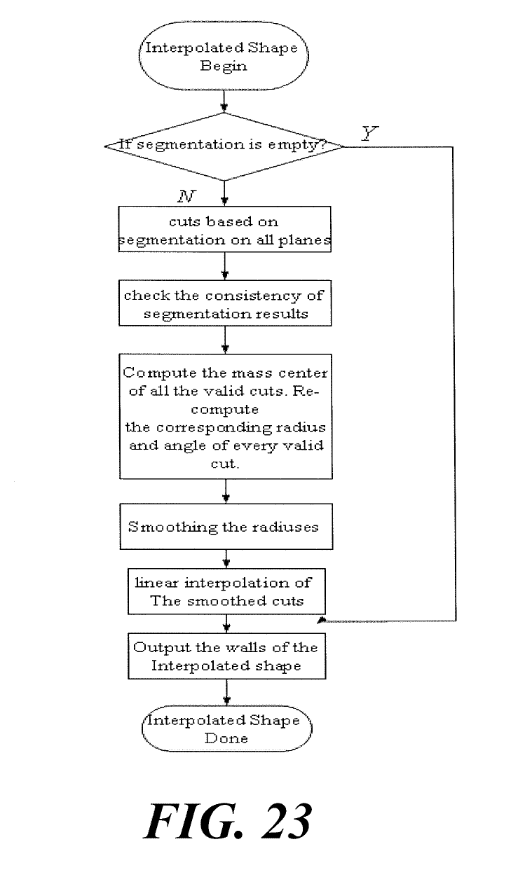

An algorithm according to an embodiment to generate the final C-mode view shape is illustrated in FIG. 23:

1) Cuts based on segmentation on all planes

In this step, extract the left most and right most cuts on each plane based on the segmentation.

2) check the consistency of the segmentation results

Theoretically, a bladder in the bladder scan is a single connected 3D volume. Due to various reasons (one of which is the segmentation algorithm searches for bladder wall blindly plane by plane), there may be more than one 3D regions and the corresponding bladder walls are also stored in the segmentation results. This step may make a topological consistency checking to guarantee that there is only one connected region in the C-mode view.

3) Compute the mass center of all the valid cuts. Re-compute the corresponding radius and angle of every valid cut. Then smooth the radius.

Compute the Cartesian coordinates for each valid cut and get the mass center. Based on this mass center, compute the corresponding radius and angle of very valid cut. Sort the new angles in ascending order. At the same time align the corresponding radius. In order to smooth the final interpolated shape, an embodiment averages the radii from above result in a pre-defined neighborhood.

4) Linear interpolation between the smoothed cuts is performed.

5) Output the walls of the interpolated shape

In an embodiment, the final output which is used to represent the interpolated shape is stored in two arrays, the size of which is 250. The dimension of the final display is on a 2D matrix, 250 by 250. The two arrays store the upper wall and lower wall location in each column respectively.

As discussed elsewhere herein, we introduced a pseudo C-mode view of the interpolated shape. An advantageous application is to provide guidance for the users to find the best scanning location and angle. This task may be called aiming.

Basically, the aiming is based on the segmentation results and it is similar as the C-mode shape functionality. In an embodiment, there are two kinds of aiming information: arrow on the probe and the intermediate shapes.

1) Arrow Feedback

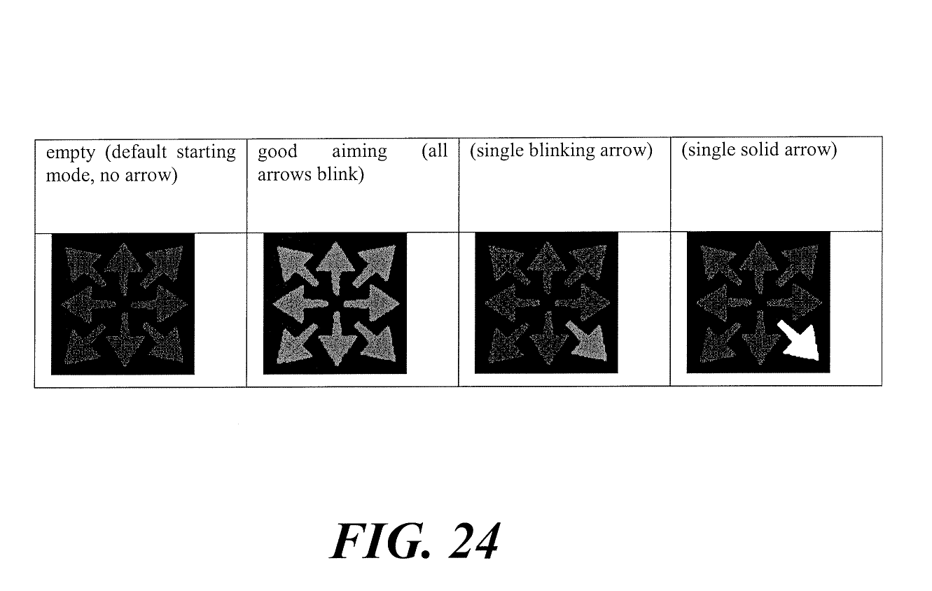

Using an extra displaying panel on the scanner, an embodiment also provides arrow feedback after a full scan. The arrow feedback may be based on the C-mode view shape. There may be four different arrow feedback modes as illustrated in FIG. 24.

Eight arrows may be used. The arrow to be used is determined by the location of the mass center of the interpolated shape in C-mode view. Based on the vector between ultrasound cone center and the mass center, the corresponding angle can be computed in a range from −180 degree to +180 degree. The [−180 180] range is divided into eight parts and each part corresponds to each arrow.

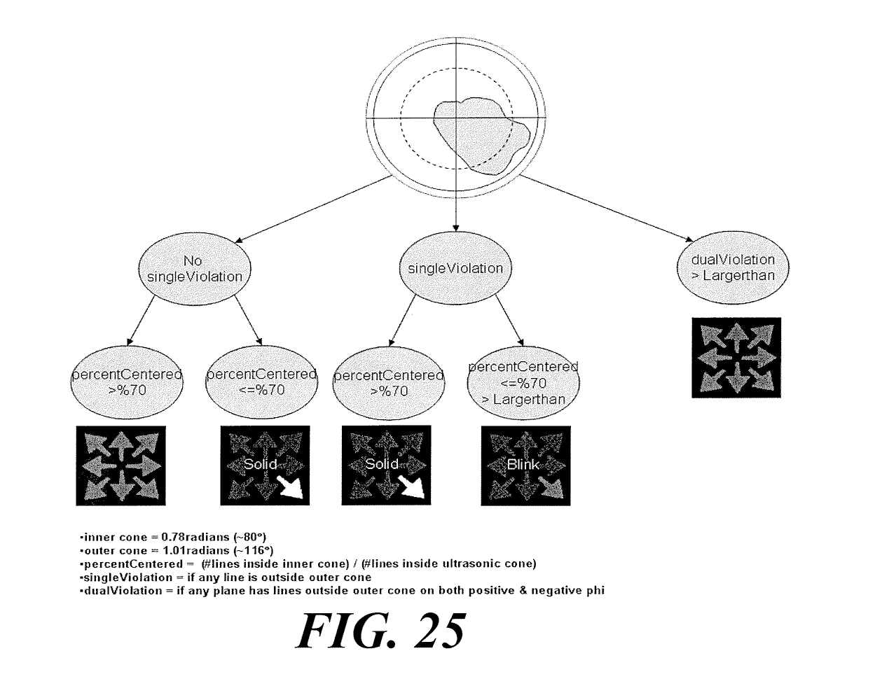

FIG. 25 illustrates rules for arrow-feedback display.

2) Pubic Bone Detection

In order to provide accurate aiming feedback information, the shadow caused by the pubic bone should also be considered. In the ultrasound image, the only feature associated with the pubic bone is the big and deep shadow. If the shadow is far from the bladder region we are interested in for volume calculation, there is no need to use this information. However, if the shadow is too close to the bladder region, or the bladder is partly inside the shadow caused by pubic bone, the corresponding volume determination will be greatly influenced. If the bladder walls are incomplete due to the shadow, we will underestimate the bladder volume.

Therefore, if the user is provided with the pubic bone information, a better scanning location can be chosen and a more accurate liquid volume measurement can be made.

An embodiment includes the following method to effect pubic bone detection based on the special shadow behind it.
On each plane, extract the left most and right most location with valid bladder wall, WL and WR. If there is no bladder walls on current plane or the wall width is too small, exit; else go on.
Compute the average frontwall depth ave_FW.
Determine the KI_threshold based on the whole image
From WL->0 searching for the shadow which is higher than ave_FW+searching range, if there are more than N shadow lines in a row, record the shadow location WL_S
From WR->nScanlines searching for the shadow which is higher than ave_FW+searching range, if there are more than N shadow lines in a row, record the shadow location WR_S
On one plane, it is only possible to have the pubic bone on one side of the bladder region. The starting location of the shadow is used to choose the most probable location for pubic bone.
Combine all valid shadow information and generate the location for pubic bone displaying

In the above procedure, a factor is to determine the KI_threshold based on the B-mode images. An embodiment utilizes an automated thresholding technique in image processing, the Kittler & Illingworth thresholding method. See, Kittler, J., Illingworth, J., 1986, Minimum Error Thresholding, Pattern Recognition, 19, 41-47.

In one instance, the shadow does not affect the volume measurement since the pubic bone is far from the bladder region; in a second case, the influence is strong since the pubic bone blocks the bladder region partly. Using a pubic icon (not shown) on the feedback screen, operators are trained to recognize when a new scanning location should be chosen and when not.

3) Intermediate Shape

Referring to FIG. 8, the step 1.6 is to show the C-mode shape. The difference between this step and the final C-mode shape is that this step only uses the grading information from the previous planes and gives instant response to the operator of current scanning status during a full scan.

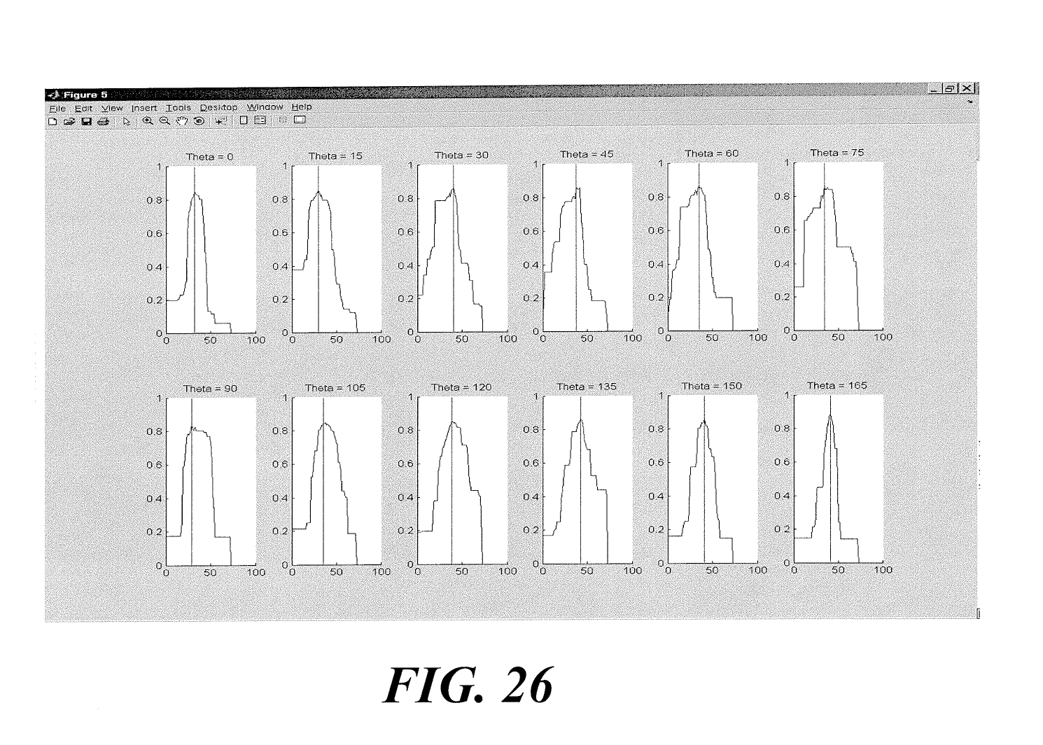

The first step is to use the grading values to find the cuts on current plane:
For each plane, there are nScanLines gradings for all lines from previous step.
Find the peak value and the corresponding line index.
Special smoothing.
Find the cuts on each plane: the left and right most line indices with grading values larger than a pre-specified threshold. [default threshold is 0.5]. An example of the gradings for all lines in an exemplary data set is displayed in FIG. 26.

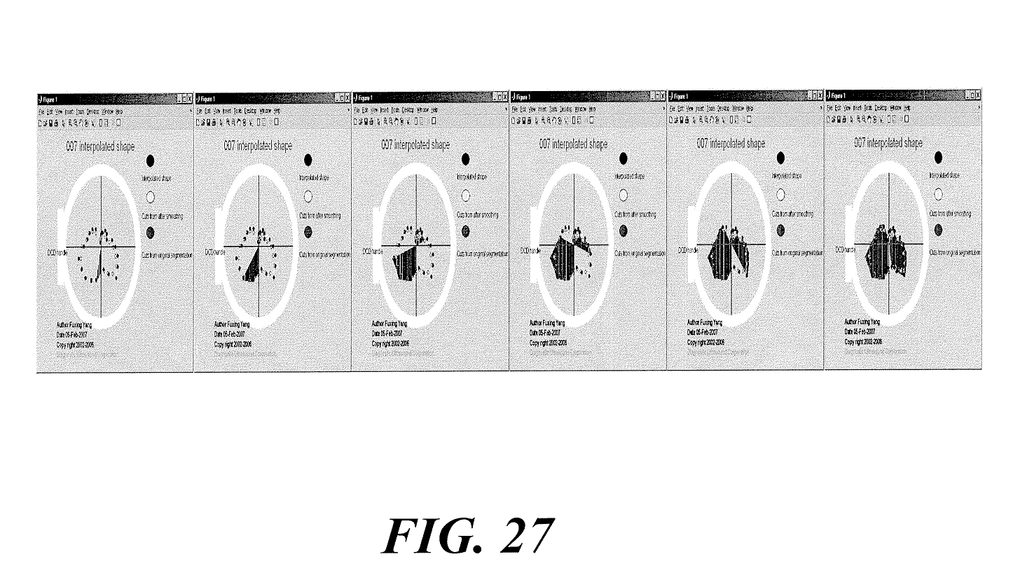

The second step is to generate a virtual painting board and draw line between the cuts on current plane and cuts from previous plane. We show a series of intermediate C-mode shapes on the exemplary data set in FIG. 27. The shapes were generated after plane 2, 4, 6, 8, 10 and 12 were collected respectively.

Reverberation Noise Control

Before an embodiment calculates the bladder volume based on the detected front and back walls, another extra step may be made to remove the wrong segmentation due to strong reverberation noise.

An embodiment has the advantage over previous approaches in that the grading information will help find the bladder lines as completely as possible. In previous approaches, bladder wall detection will stop early when strong reverberation noise is present.

However, even the above improvement is still not able to fix the wrong segmentation on some lines due to reverberation noises. Therefore, an embodiment includes the following method to remove the small wedges on the bladder walls using shape information:
For each plane
For each line
If there is fw on current line, search for the nearest fw on the left, which has a fw valid_FW_change shallower than current fw; search for the nearest fw on the right, which also has a fw valid_FW_change shallower than current fw. If the searching is successful on both sides, we use the found fw pair to generate a new fw at current location.
If there is bw on current line, search for the nearest fw on the left, which has a bw valid_BW_change shallower than current bw; search for the nearest bw on the right, which also has a bw valid_BW_change shallower than current bw. If the searching is successful on both sides, we use the found bw pair to generate a new bw at current location.
End of each line
End of each plane

An embodiment includes an interpolation approach using adjacent bladder wall shape. We have already considered the cases when the bladder shape is indeed with large convex part on the front or back wall by defining two parameters (valid_FW_change and valid_BW_change).

Volume Measurement

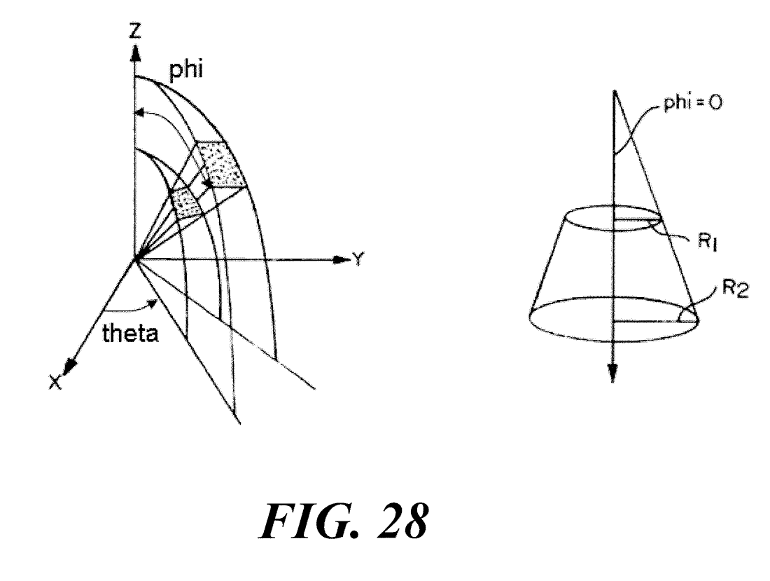

Referring now to FIG. 28, in an embodiment, in order to compute the bladder volume, the following information may be used:
Spherical coordinate phi and theta
The axial front wall and back wall locations
Axial resolution

For every scan line except the broadside scan line (phi=0), a spherical wedge shape is defined, with the physical scan line passed through the center of the wedge. The spherical wedge is bounded on top by the front wall and on the bottom by the back wall, on the sides by the average of the current scan line spherical angles and the next closest spherical angles. [The left-side image in FIG. 28.]

For broadside scan line, a truncated cone is used. [The right-side image in FIG. 28.]

While the particular embodiments have been illustrated and described for presenting color-coded ultrasound images based upon ultrasound harmonic frequencies exhibiting optimal signal-to-noise ratios for sub-structures, many changes can be made without departing from the spirit and scope of the invention. For example, using harmonics in imaging applications other than ultrasound may be employed. Additionally, although estimations applied to urine content have been emphasized herein throughout, embodiments of the invention apply to analysis of other bodily fluids, such as amniotic fluid and blood, as well. For example, amniotic fluid volume in a pregnant female can be measured by employing at least one embodiment of the invention. The non-pregnant female's uterus can be distinguished from a bladder by employing at least one embodiment of the invention, inasmuch as blood occasionally present within the uterus of the non-pregnant female does not have as high a Goldberg number as amniotic fluid in the pregnant female or urine within the female bladder, in either case. As such, for example, blood in an engorged umbilical cord may be distinguished from amniotic fluid by employing at least one embodiment of the invention. Accordingly, the scope of embodiments of the invention is not limited by the disclosure of the particular embodiments. Instead, embodiments of the invention should be determined entirely by reference to the claims that follow.

The embodiments of the invention in which an exclusive property or privilege is claimed are defined as follows:

1. A system, comprising:
at least one transducer configured to transmit at least one ultrasound pulse into a region of interest (ROI) of a patient, the pulse having at least a first frequency, the pulse propagating through a bodily structure in the ROI;
at least one receiver configured to receive at least one echo signal corresponding to the pulse, the at least one echo signal having the first frequency and at least one harmonic multiple of the first frequency; and
a processor configured to automatically determine, from the at least one harmonic multiple, at least one boundary of the bodily structure.

2. The system of claim 1 wherein the bodily structure comprises a bladder.

3. The system of claim 1 wherein the bodily structure comprises a heart.

4. The system of claim 1 wherein the at least one transducer is further configured to transmit multiple ultrasound pulses through multiple scan planes; and
wherein the at least one boundary of the bodily structure is determined from echo signals corresponding to the multiple pulses.

5. The system of claim 1 wherein the processor is further configured to automatically determine, from the at least one harmonic multiple, an amount of fluid within the bodily structure.

6. The system of claim 1 wherein determining the at least one boundary comprises determining a Goldberg number associated with the at least one echo signal.

7. The system of claim 1 wherein the processor comprises a neural network.

8. A system, comprising:
at least one transducer configured to transmit at least one ultrasound pulse into a region of interest (ROI) of a patient, the pulse having at least a first frequency, the pulse propagating through a bodily structure in the ROI;
at least one receiver configured to receive at least one echo signal corresponding to the pulse, the at least one echo signal having the first frequency and at least one harmonic multiple of the first frequency; and
a processor configured to automatically determine, from the at least one harmonic multiple, an amount of fluid within the bodily structure.

9. The system of claim 8 wherein the bodily structure comprises a bladder.

10. The system of claim 8 wherein the fluid comprises urine.

11. The system of claim 8 wherein the processor is further configured to automatically determine, from the at least one harmonic multiple, at least one boundary of the bodily structure.

12. The system of claim 8 wherein the at least one transducer is further configured to transmit multiple ultrasound pulses through multiple scan planes; and
wherein the amount of fluid is determined from echo signals corresponding to the multiple pulses.

13. A method for ultrasonic imaging of a region-of-interest within a subject, comprising:
exposing the region-of-interest with ultrasound energy delivered from an ultrasonic transceiver emitting a fundamental ultrasound frequency acoustically coupled and placed against a first surface location of the subject;
collecting ultrasound echoes by the ultrasonic transceiver from structures located in the region-of-interest;
discerning a plurality of harmonic frequencies within the ultrasound echoes;
selecting a harmonic frequency from the plurality of harmonic frequencies;
detecting a structure within the region-of-interest using the selected harmonic frequency;
presenting a color-coded image of the structure on a display in proportion to the strength of the signals of the selected harmonic frequency; and
determining positional information of the structure in relation to the region-of-interest with regard to the first surface location of the subject.

14. The method of claim 13, wherein exposing the region-of-interest includes repositioning the transceiver in relation to the region-of-interest from the positional information determined from the structure using the selected harmonic frequency and re-exposing the structure with the fundamental frequency.

15. The method of claim 14, wherein the positional information is determined from algorithms executed by a computer readable medium operated by a microprocessor device in signal communication with the display and the transceiver.

16. The method of claim 15, wherein the positional information of the structure is conveyed to directional indicators associated with the transceiver to direct a user to a second surface location of the subject to reposition the transceiver for re-exposing the region-of-interest with the fundamental frequency.

17. The method of claim 13, wherein discerning the plurality of harmonic frequencies includes a second harmonic frequency and a third harmonic frequency.

18. The method of claim 17, wherein selecting the harmonic frequency includes determining a signal-to-noise ratio of the second harmonic frequency and the third harmonic frequency arising from structural components within the structures having differing echogenic and ultrasound energy attenuating characteristics.

19. The method of claim 18, wherein the signal-to-noise ratio includes signal-to-noise ratios exhibited by echogenic and non-echogenic structural components.

20. The method of claim 19, wherein presenting the color-coded image includes color assignments to pixels defining the echogenic and non-echogenic structural components.

21. A system for ultrasonic imaging of a region-of-interest within a subject, comprising:
an ultrasound transceiver configured to deliver ultrasound pulses having a fundamental frequency to and acquire ultrasound echoes returning from structures within the region-of-interest;
a microprocessor device in signal communication with the transceiver;
a display in signal communication with the microprocessor device and the transceiver; and
a computer readable medium having algorithms configured to detect, analyze, and select an ultrasound harmonic frequency suitable for detecting and presenting the structures in a color-coded image on the display.

22. The system of claim 21, wherein the algorithms include sub-algorithms configured to assign color shades to image pixels defining echogenic and non-echogenic structural components of the structures.

23. A method, comprising:
transmitting, with at least one transducer, at least one ultrasound pulse into a region of interest (ROI) of a patient, the pulse having at least a first frequency, the pulse propagating through a bodily structure in the ROI;
receiving, with at least one receiver, at least one echo signal corresponding to the pulse, the at least one echo signal having the first frequency and at least one harmonic multiple of the first frequency; and
automatically determining, from the at least one harmonic multiple, at least one boundary of the bodily structure and an amount of fluid within the bodily structure.

24. The method of claim 23 wherein the bodily structure comprises a bladder.

25. The method of claim 23 wherein the fluid comprises urine.

26. The method of claim 23 wherein the processor is further configured to automatically determine, from the at least one harmonic multiple, at least one boundary of the bodily structure.

27. The method of claim 23 wherein the at least one transducer is further configured to transmit multiple ultrasound pulses through multiple scan planes; and
wherein the boundary and amount of fluid are determined from echo signals corresponding to the multiple pulses.
